(19) United States
(12) Patent Application Publication
(10) Pub. No.: US 2026/0337651 A1
(43) Pub. Date: Oct. 29, 2026

(54) CELL-CULTIVATED MILK FOR CONFECTIONERY AND DAIRY PRODUCT USES

(71) Applicant: SENARA B.V., Maastricht (NL)

(72) Inventor: Svenja DANNEWITZ PROSSEDA, Freiburg (DE)

(21) Appl. No.: 19/700,133

(22) Filed: Jun. 5, 2026

Related U.S. Application Data
(63) Continuation of application No. PCT/IB2024/062345, filed on Dec. 6, 2024.
(63) Continuation-in-part of application No. 18/881,178, filed on Jul. 4, 2023.
(60) Provisional application No. 63/358,464, filed on Jul. 5, 2022.
(60) Provisional application No. 63/608,140, filed on Dec. 8, 2023.

(51) Int. Cl. C12P 19/12; A23C 9/12; A23G 3/46; C12N 5/071

(52) U.S. Cl. CPC C12P 19/12; A23C 9/1203; A23G 3/46; C12N 5/0631

(57) ABSTRACT

Cell-cultured milk generated in vitro in a reactor assembly is employed in the production of food products including chocolate, confections, ice cream, cheese, yogurt, cream, and non-food products such as pharmaceuticals, cosmetics and plastics. The processing used to generate the cell-cultured milk can be tailored to the specific type of product, including nutrient enhancement for food products and removal of undesirable components of traditional dairy milk.

### RELATED APPLICATIONS

This is a continuation under 35 U.S.C. 365 (c) of International Application No. PCT/IB2024/062345, filed Dec. 6, 2024, which claims the benefit of the priority of U.S. Provisional Application No. 63/608,140, filed Dec. 8, 2023, and is also a continuation-in-part of U.S. application Ser. No. 18/881,178, filed Jan. 3, 2025, which is a national stage under 35 U.S.C. 371 of International Application No. PCT/IB2023/056912, filed Jul. 4, 2023, which claims the benefit of the priority of U.S. Provisional Application No. 63/358,464, filed Jul. 5, 2022, each of which is incorporated herein by reference in its entirety.

### FIELD OF THE INVENTION

The present invention relates to in vitro milk production and more particularly to cell-cultivated milk for use in the production of milk-based products including confectionary items such as chocolate and ice cream, and dairy products such as cheese, yogurt, and cream, and non-food products.

### BACKGROUND

The demand for worldwide milk production continually increases in response to a range of factors, including global population growth and changing, i.e., westernization, of diets in Asian countries. Worldwide consumption of dairy is projected to increase by over $100B over the coming decade. This, in turn, is placing increasing pressure on natural resources, increasing animal welfare concerns, and leading to increased deforestation alongside greater production of greenhouse gases. Dairy consumption in the U.S. accounts for about 2% of the country's greenhouse gas emissions. To meet the worldwide demand, around 270 million dairy cows are farmed every day. Every component in the milk has been associated with an array of both positive and negative health effects.

Milk is a complex colloidal matrix that contains milk fat, lactose, and a number of different components, including naturally occurring hormones. The sensory and functional characteristics of milk stem from its micronutrient profile that has proven costly and difficult to replicate. In general, the gross composition of cow's milk in the U.S. is 87.7% water, 4.9% lactose (carbohydrate), 3.4% fat, 3.3% protein, and 0.7% minerals. Milk composition varies depending on the species (cow, goat, sheep), breed (Holstein, Jersey), the animal's feed, and the stage of lactation.

Studies have shown that the fat and protein content of milk is based on the breed of cattle. Holsteins have the lowest fat and protein content, while Jersey and Guernsey breeds have the highest fat and protein content. Even within a single herd, the milk protein can range from 1.57% to 4.66%, with an average of 3.05%; while the milk fat ranges from 1.77% to 5.98%, with an average of 3.76%. The food source, temperature, humidity, and seasonality all played important roles to the variation of milk quality in milk production. To address variations in milk composition, a common practice is to blend the milk from different cows together in bulk tanks, producing a relatively consistent composition of milk year round in the U.S.

Another issue with milk quality is the presence of naturally occurring hormones found in milk. These hormones include Prolactin (15.4±1 ng/mL), IGF-1 (4±1 ng/ml), PGE2 (2.4±0.3 ng/ml), PGF2α (2±0.5 ng/ml), TXB2 (1±0.5 ng/ml), Corticosteroids (14±4 ng/ml), Testosterone (0.09±0.03 ng/ml), 5α-esteroids (3±1 ng/mL), Progesterone (12±2 ng/ml), Esterone (0.13 ng/ml), 17β-estradiol (0.02 ng/mL), Esteriol (0.027=0.01 ng/mL). Additionally, hormones and other foreign agents, such as antibiotics and pharmaceutics, are often present in industrial-scale milk arising from the need to treat an entire herd for infections during their milk production (i.e., chronic udder infections).

While there is little debate about the presence of physiologic concentrations of these hormones, the potential biological effects of such hormones on animals and humans may not be well understood and can be profound. Further, the presence of hormones can raise significant safety concerns about dairy foods, especially steroid hormones like estrogens. Some evidence suggests potential links with breast and prostate cancers. Accordingly, special attention is warranted, especially during major developmental periods, e.g., perinatal and pubertal periods.

To this end, and with respect to the considerable progress in developing of analytical methods and bioassays, it is important to clarify the potential impact of the presence of hormones, especially steroid hormones like estrogens, when they are a common component of diets and being consumed at a regular basis for years.

Dietary alternatives to dairy products are found in plant-based milk and milk products. Milk from plants such as almond, soy, cashew, and oat lack the naturally occurring hormones found in animal-based milk and are becoming increasingly popular as a good source of protein without raising the issues of dairy intolerance that many people experience. However, the plant-based milks from these alternative sources tend not to be as effective in providing the functionality of animal-based milk that translates to production of other dairy products such as cheese, butter, and yogurt. Furthermore, the increased popularity of plant-based milks has brought to light other sustainability concerns, for example, the widespread deforestation that has been employed to expand soy production, or the large amounts of water required for almond production in areas experiencing long-term drought.

Another area of interest within the dairy business is the alternative milk product called “A2 milk”. Cow's milk generally contains two types of β-casein proteins: A1 and A2 types, which differ in amino acid histidine or proline, respectively, at position 67 of the protein structure. The hypothesis is that traditional bovine milk that contains A1 β-casein proteins may contribute to negative health effects, including digestive discomfort, intestinal inflammation, and milk allergies, whereas A2 β-casein does not appear to induce these effects.

Both the A1 β-casein and the A2 β-casein proteins contain 209 amino acids (AA). However, upon digestion, the A1 β-casein releases β-casomorphin-7 (BCM-7), which triggers a cascade of events that increase inflammation and gastrointestinal discomfort. Therefore, consumption of milk containing A1 β-casein is hypothesized to be associated with increased gastrointestinal inflammation, worsening of PD3 symptoms, delayed transit, and decreased cognitive processing speed and accuracy. A recent study confirmed that subjects consuming A1/A2 β-casein milk presented greater digestive symptoms associated with lactose intolerance, whereas the consumption of A2 β-casein milk was not found to aggravate these symptoms. Inflammatory markers, such as IL-4, IgG, IgE, and IgG1, were significantly lower in A2 β-casein milk consumers. Currently, no economic solutions to this A1 β-casein issue have been developed due to the close similarity of the two types of β-casein, A1 and A2 types and as cows producing milk rich in the A1-protein tend to belong to the breeds that produce milk in high quantities (high-producing cows), for example Holstein cows.

Milk and dairy products, including cream, butter, yogurt, and cheese, constitute a significant portion of the human diet, providing important sources of protein, vitamins, and minerals. For many people, dairy is the easiest way to obtain supplementary nutrients to keep the heart, muscles, and bones healthy and functioning properly. However, the milk quality related issues discussed above have plagued the dairy industry for many years and urgently need a solution.

The need for improvements to milk production technology is apparent, driven by several factors including the current unsustainable approach to producing milk through cattle farming (inefficient use of land and agricultural resources, greenhouse gas production, and more); the lack of comparable alternatives to milk; and the limited sources of consistent quality and high-quality milk.

U.S. Pat. No. 11,236,299 (assigned to Biomilk Ltd. (Rehovot, IL)), incorporated herein by reference, discloses a method for in vitro milk production by employing an array of mammary organoids seeded on tertiary-branched, resilient duct scaffolding. In an exemplary implementation, the milk production system includes an array of vessels, each vessel comprising a plurality of mammary organoids (MO); a nutrient supply reservoir operable to feed each vessel and a milk collection module. The key component of the system is the MOs, which are the mammary epithelial cells forming multicellular three-dimensional structures (mammary organoids) embedded in matrix. Upon seeding of the scaffolding with the MO, estrogen and progesterone are instrumental in inducing growth and morphogenesis of epithelium via induction of paracrine signaling between mammary stroma and epithelium comprising the seeded MOs. The MOs then start secreting milk for a period of between 10-21 days in a medium containing prolactin, nutrients, and growth factors. While the Wilk patent discloses a method for in vitro production of milk, it does not provide a range of options for modifying the milk product in terms of cellular structure so as to enhance the nutritional content, reduce the reliance on hormones, select the preferable A2 β-casein, and other “designer milk” products, e.g., hypoallergenic milk, that will serve to provide a more sustainable and nutritional food source to meet growing worldwide demand.

In view of growing demand for dairy products, a more sustainable and versatile approach to milk production, to overcome the environmental and animal welfare challenges faced by the current dairy industry is needed. The use of traditional milk in confectionery and dairy products has been widespread. However, limitations such as environmental impact, animal welfare concerns, and supply chain issues necessitate innovative alternatives. Cell-cultivated milk presents a sustainable and ethically viable solution, revolutionizing the production of various food items.

### SUMMARY

According to embodiments of the invention, a system and method are provided for in vitro milk production using mammary cells cultured for enhanced cell proliferation and milk and milk-component secretion. Enhancement of cell proliferation is achieved at least in part through mechanical stimulation via the application of flow to the culture medium and alteration of flow through a variety of different methods, including, but not limited to, unidirectional laminar flow, turbulent flow, pulsatile flow, and oscillating flow. The constant mechanical stimulation provided by the inventive system enables the production of healthy epithelial cells (ECs) and production of milk that is substantially hormone-free (other than prolactin). The system and method disclosed herein are generally applicable to production of cellular proteins and dairy for food culture and for bioindustrial use.

In one aspect of the invention, an apparatus for in vitro milk production includes: a reactor assembly comprising: an elongated shell configured for retaining an extracellular medium; one or more hollow tube disposed within the extracellular medium generally in longitudinal alignment within the shell, the one or more tube having an inlet end, an outlet end, and an inner surface configured for attaching a monolayer of lactating cells, wherein at least a portion of the the hollow tube is formed from a semipermeable material configured to permit diffusion of the extracellular medium into the one or more tube, wherein the extracellular medium comprises a nutrient solution for maintaining the lactating cells; a pump in fluid connection with the one or more tube, the pump configured to generate a pulsatile or oscillating flow of a lactating medium through the hollow tubes to induce a shear stress on the lactating cells, wherein the pump is controlled to alternate among different shear stress levels within a predetermined range, wherein variation of the shear stress stimulates the lactating cells to produce a milk product; a supply loop for supplying and circulating the extracellular medium through the shell; and a reservoir in fluid connection with the outlet end of the one or more tube, the reservoir configured for collecting the milk product. The apparatus may further include a feedback loop disposed near the outlet ends of the one or more tube for recirculating the milk product into the inlet end for further enrichment. In some embodiments, the predetermined range of shear stress levels is 2 to 75 dyn/cm². The supply loop may include a reservoir configured for removing used extracellular medium and adding fresh extracellular medium.

In some embodiments, the lactating cells are co-cultured with feeder cells. The feeder cells may be peripheral blood mononuclear cells. In some embodiments, the lactating cells may be isolated from the milk of healthy cows. In other embodiments, the lactating cells may be extracted from healthy mammary tissue identified using a panel of biomarkers selected from CD9, CD47, CD54, CD59, CD95, CD164, CD49b, CD66, CD24, CK8/18, CK19, MUC1, GATA3, EPCAM, CD13, CD15s/CD73/CLA, +CD166/CD227/CD340, CD10, CD90, CD200, CD29/CD142/CD271, CK5, CK14, CK17, SMA, VIM, CD34/CD39/CD140b, and CD49e. In some embodiments, the lactating cells may be extracted from healthy mammary tissue selected from all breast cells, all breast epithelial cells, breast luminal cells, breast luminal progenitor cells, mature breast luminal cells, breast myoepithelial cells, and breast stromal cells. The lactating cells may be genetically modified to induce hyperlactation, or to produce milk that is one or more of hypoallergenic, reduced lactose, and has increased A2 β-casein proteins. In some embodiments, each hollow tube may be coated with collagen. The one or more tube is formed from a material at least a portion of which is a semi-permeable capillary membrane. In some embodiments, the inner surface of the one or more tube is first bound with a layer of a support matrix, wherein the lactating cells are attached to a non-bound surface of the support matrix, and wherein the lactating cells form a monolayer of the lactating cells on top of the support matrix.

In some embodiments, the apparatus may further include a system controller configured to generate control signals to the pump and the supply loop.

An optional mechanical stimulation assembly may be included to apply a squeezing force to an exterior of the one or more tube in a direction from the inlet end toward the outlet end. An optional one or more light source may be disposed within the reactor assembly to expose the lactating cells to light stimulation. In some embodiments, the one or more light source is a light emitting diode (LED) that emits light at 450 nm.

The lactating medium may include one or more of an EpiCult™ Plus medium, a Lab-grown FCS alternative medium, and a FCS free medium, and may further include prolactin.

In another aspect of the invention, a milk production facility may be constructed by interconnecting a plurality of the above-described apparatuses.

In still another aspect of the invention, a method of in vitro milk production in the above-described apparatus includes: supplying the one or more tube with the lactating medium, wherein the lactating medium comprises prolactin; forming a monolayer of lactating cells attached on the inner surface of the one or more tube; and controlling the pump to alternate among different shear stress levels within a predetermined range, wherein variation of the shear stress levels during a processing period stimulates the lactating cells to produce a milk product. The method may further include recirculating the milk product via a feedback loop into the one or more tube to enrich the milk product until the predetermined milk quality is achieved In some embodiments, the lactating cells are co-cultured with feeder cells. The feeder cells may be peripheral blood mononuclear cells. The lactating medium may be one or more of an EpiCult™ Plus medium, a Lab-grown FCS alternative medium, and a FCS free medium

The lactating cells may be isolated from the milk of healthy cows in some embodiments. In other embodiments, the lactating cells may be extracted from healthy mammary tissue identified using a panel of biomarkers selected from CD9, CD47, CD54, CD59, CD95, CD164, CD49b, CD66, CD24, CK8/18, CK19, MUC1, GATA3, EPCAM, CD13, CD15s/CD73/CLA, +CD166/CD227/CD340, CD10, CD90, CD200, CD29/CD142/CD271, CK5, CK14, CK17, SMA, VIM, CD34/CD39/CD140b, and CD49e. In other embodiments, the lactating cells may be extracted from healthy mammary tissue selected from all breast cells, all breast epithelial cells, breast luminal cells, breast luminal progenitor cells, mature breast luminal cells, breast myoepithelial cells, and breast stromal cells.

The lactating cells may be genetically modified to induce hyperlactation and/or to produce milk that is one or more of hypoallergenic, reduced lactose, and has increased A2 β-casein proteins.

In further embodiments, the cell-cultivated milk disclosed herein can be processed for production of conventional milk-based products, either integrated with the primary milk processing sequence or via post-processing of the basic milk. Such embodiments harness cell-cultivated milk, derived from cultured cells rather than livestock, to create a range of confectionery and dairy products, including, but not limited to, chocolate, confections, baked goods, ice cream, butter, cheese, yogurt, and cream. The method offers reduced environmental impact, addresses animal welfare concerns, and ensures a consistent and scalable milk supply for production of a wide range of milk-based foods and non-food products.

### DESCRIPTION OF THE DRAWINGS

Those of skill in the art will understand that the drawings, described below, are for illustrative purposes only. The drawings are not intended to limit the scope of the present teachings in any way.

FIG. 1A is a diagrammatic view of the basic components of the inventive bioreactor according to one embodiment; FIG. 1B diagrammatically illustrates elements within a hollow tube that simulate the native environment for supporting a monolayer of co-cultured alveolar cells and feeder cells attached to the inner lumen of the tube; FIG. 1C is a diagrammatic view with tube cross-sections showing monolayers of lactating cells attached to the inner surface of the hollow fibers; FIG. 1D illustrates laminar flow in the hollow fiber bioreactor; FIG. 1E is a diagrammatic view of an alternative tube configuration.

FIGS. 2A-2C diagrammatically illustrate another embodiment of the inventive bioreactor, where FIGS. 2A and 2B provides a front and side views, respectively, of an embodiment of a bioreactor assembly; FIG. 2C shows a bottom view of the assembly with a cross-section of the cartridge and a 3-D image of the hollow tubes; FIG. 2D illustrates an exemplary layout of a bank of bioreactors in a milk production facility.

FIG. 3 diagrammatically illustrates a model of the formation of a monolayer of co-cultured alveolar cells and feeder cells attached to the inner lumen of the hollow fiber or hollow tube bioreactor. The mammary epithelium consists of two differentiated cell types organized into two cell layers. An inner layer of luminal epithelial and an outer layer of myoepithelial cells in direct contact with the basal membrane. Prompted by the hormone prolactin, the alveoli take up nutrients from the blood supply and produce breast milk.

FIG. 4 compares effects of different types of mechanical stimulation by the flow, Shear Stress vs Shear Strain, to the endothelial cells for healthy EC cells. The lower panel provides SEM micrographs of EC cells with and without exposure to shear stress.

FIGS. 5A-5B are photomicrographs of mammary epithelial cells possessing an epithelial-like phenotype and mammary myoepithelial cells possessing a spindle-like phenotype, respectively; FIG. 5C shows growth rate curves of breast tissue cell lines 1×10⁴ cells were seeded and measurements were subsequently taken on the 4 following days. Values for three independent cell samples for each day were compared by using unpaired t-tests. Significance between mammary epithelial cells (green) and mammary myoepithelial cells (red) cell growth rates was thereby determined for day 3 (p=0.007) and day 4 (p=0.0228). These values are highlighted in the diagram through asterisks; FIGS. 5D and 5E are plots showing co-culture PBMC-conditioned media growth rates.

FIG. 6 is a diagram of the induction of hyperlactation in cells by CRISPR.

FIGS. 7A and 7B illustrate exemplary process flows for processing of bulk chocolate and production of chocolate candy, respectively.

### DETAILED DESCRIPTION OF EMBODIMENTS

To facilitate understanding of the invention, a number of terms and abbreviations as used herein are defined below as follows:

As used herein, the term “amino acids” refers to the molecular basis for constructing and assembling proteins, such as enzymes. Peptide bonds (i.e., polypeptides) are formed between amino acids and assemble three-dimensionally (3-D). The 3-D assembly can influence the properties, function, and conformational dynamics of the protein. Within biological systems, the protein may: (i) catalyze reactions as enzymes; (ii) transport vesicles, molecules, and other entities within cells as transporter entities; (iii) provide structure to cells and organisms as protein filaments; (iv) replicate deoxyribonucleic acid (DNA); and (v) coordinate actions of cells as cell signalers.

As used herein, the term “nucleotides” refers to the molecular basis for constructing and assembling nucleic acids, such as DNA and ribonucleic acid (RNA). There are two types of nucleotides-purines and pyrimidines. The specific purines are adenine (A) and guanine (G). The specific pyrimidines are cytosine (C), uracil (U), and thymine (T). T is found in DNA, whereas U is found in RNA. The genetic code defines the sequence of nucleotide triplets (i.e., codons) for specifying which amino acids are added during protein synthesis.

As used herein, the term “genes” refers to regions of DNA. Amino acid sequences in the proteins, as defined by the sequence of a gene, are encoded in the genetic code.

As used herein, a recombinant nucleic acid or protein is a nucleic acid or protein produced by recombinant DNA technology, e.g., as described in Green and Sambrook (2012).

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide. The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

As used herein, the term “bioreactor” refers to vessels or tanks in which whole cells or cell-free enzymes transform raw materials into biochemical products and/or less undesirable by-products. The bioreactor is designed and operated to provide the environment for product formation, in this case, a milk product. Industrial bioreactors may be operated as batch reactors or continuously, aerobically or anaerobically, and with pure or mixed cultures. In some bioreactors, three phases (gas, liquid, and solid) are present and mass transfer can be an important consideration. Biofilms and immobilized cells can be used to retain microbial biomass in a flow bioreactor. Sensors, instrumentation, and control systems are essential for industrial bioreactors.

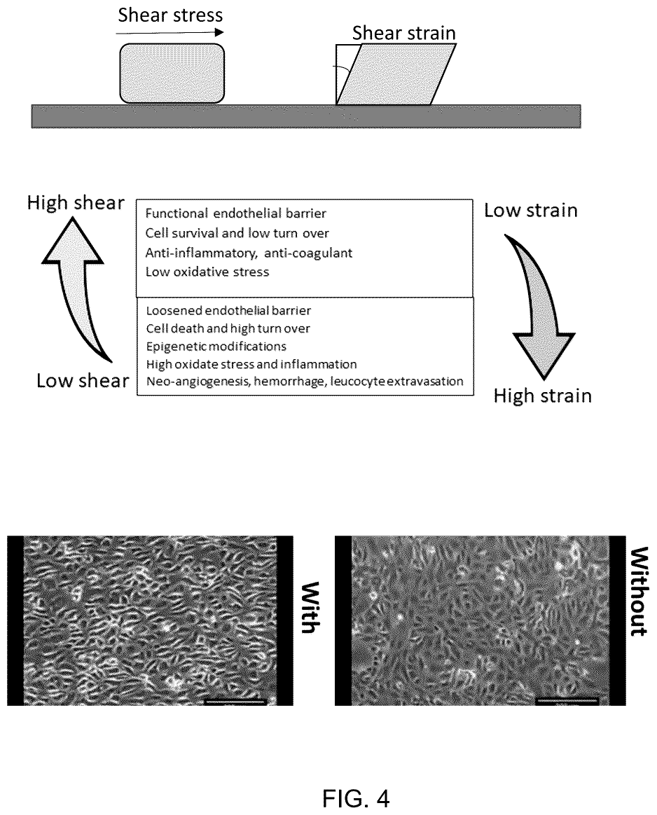

As used herein, the term “Shear Stress,” often denoted by t (Greek: tau), refers to the component of stress coplanar with a material cross section. (See, e.g., FIG. 4.) It arises from the shear force, the component of force vector parallel to the material cross section. Any real fluids (liquids and gases included) moving along a solid boundary will incur a shear stress at that boundary. The no-slip condition dictates that the speed of the fluid at the boundary (relative to the boundary) is zero; although at some height from the boundary the flow speed must equal that of the fluid. The region between these two points is named the boundary layer. For all Newtonian fluids in laminar flow, the shear stress is proportional to the strain rate in the fluid, where the viscosity is the constant of proportionality. For non-Newtonian fluids, the viscosity is not constant. The shear stress is imparted onto the boundary as a result of this loss of velocity.

As used herein, the term “Shear Strain” refers to the ratio of displacement to an object's original dimensions due to stress and is the amount of deformation perpendicular to a given line rather than parallel to it. Shear strain is a sideways force exerted on a medium and is measured as a change in angle between lines that were originally perpendicular. (See, e.g., FIG. 4.)

As used herein, the term “Pulsatile flow” refers to flow with a periodic pressure fluctuation wave traveling along the flow path. Pulsatile flow systems may mimic blood flow characteristics within the heart and vasculature system.

As used herein, the term “Effective amount” refers to those amounts that, when administered to a particular subject in view of the nature and severity of that subject's condition, will have a desired biological effect, e.g., an amount that will cure, prevent, inhibit, or at least partially arrest or partially prevent a target reaction.

A number of acronyms and abbreviations are used throughout this disclosure. Table 1 below summarizes and provides a glossary of several of the frequently used acronyms and abbreviations:

TABLE 1

| ACRONYM | DEFINITION |
|---|---|
| EC | epithelial cell |
| mEP or MEP | mammary myoepithelial and epithelial cell |
| MEC | mammary epithelial cell |
| ECM | extracellular matrix or medium |
| HDF | human dermal fibroblast |
| InC | Intercapillary |
| ExC | Extracapillary |

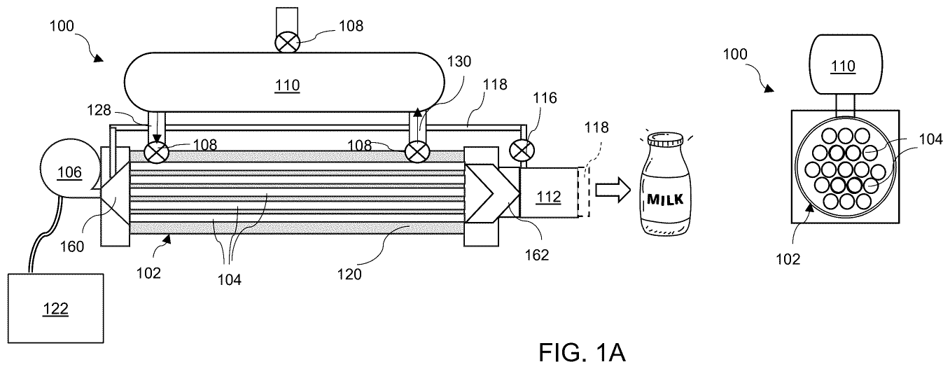

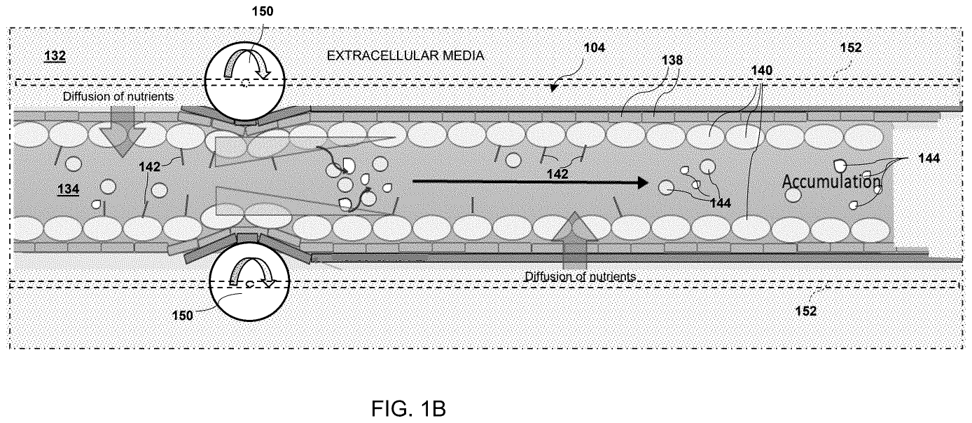

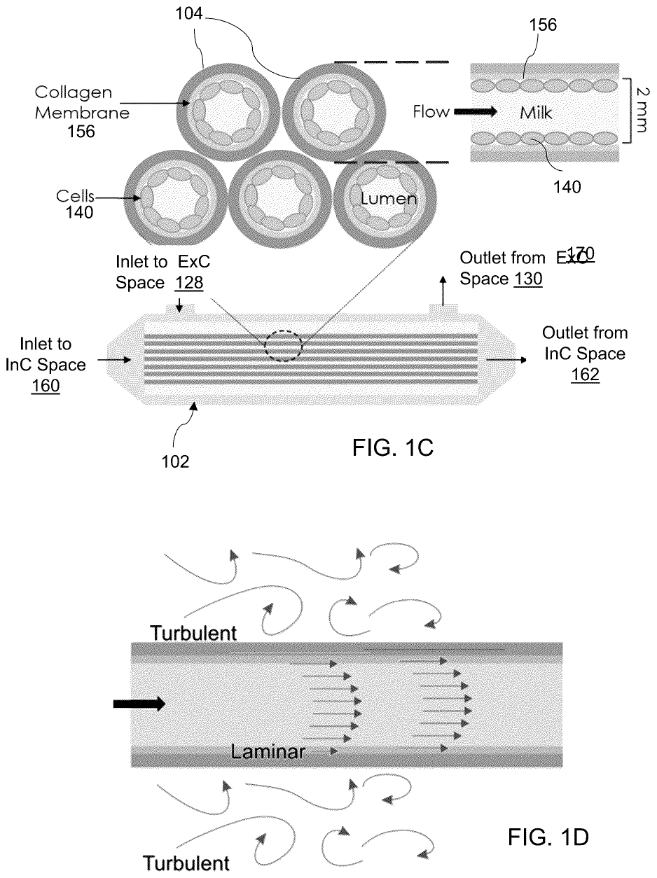

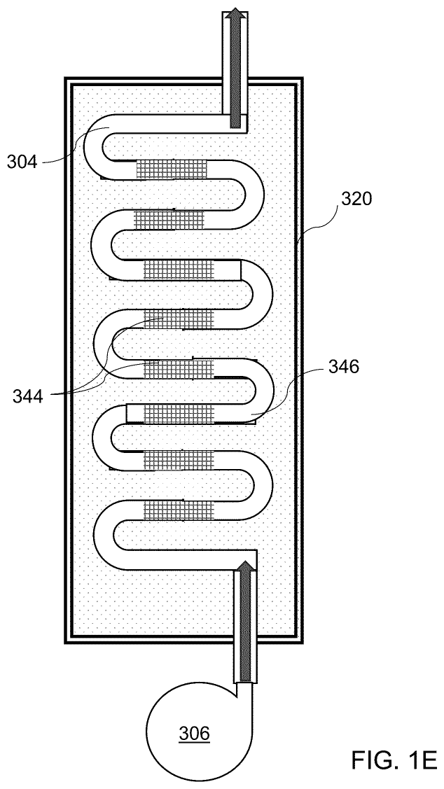

The terms “hollow tube”, “hollow tubing”, “hollow fiber”, and “lumen” may be used interchangeably through the description to refer to an elongated hollow structure through which fluid flows under pressure applied by a pumping system in fluid connection with the hollow structure. The hollow structure may be formed from a number of different materials which may be rigid, flexible, permeable, semipermeable, or non-permeable. Examples of materials include, but are not limited to, glass, acrylic, plastic, polymers, fibers, silicone, ceramic, and filter membranes. In some embodiments, a hollow structure may be compressible or contractable so as to simulate a peristaltic-like action, for example, by applying opposing rollers to an outer surface of the tubing to “squeeze” the fluid within the interior of the tubing from a first end to a second end of the tubing. (See, e.g., FIG. 1B). In other embodiments, the tube may be a hybrid assembly of sections of tube made from different materials, some of which may be permeable or semi-permeable membranes, while others may be non-permeable.

### Lactation Biology

Lactation is the process by which milk is synthesized and secreted from the mammary glands. Mammary glands are modified sweat glands, and are composed primarily of adipose and collagenous tissue, with mammary glands making up a very minor proportion of breast volume. The mammary gland is composed of milk-transporting lactiferous ducts, which expand and branch extensively in response to estrogen, growth hormone, cortisol, and prolactin. Moreover, in response to progesterone, clusters of breast alveoli bud from the ducts and expand outward toward the chest wall. Breast alveoli are balloon-like structures lined with milk-secreting cuboidal cells, or lactocytes, that are surrounded by a net of contractile myoepithelial cells. Milk is secreted from the lactocytes, fills the alveoli, and is squeezed into the ducts. Clusters of alveoli that drain to a common duct are called lobules; the lactating female has 12-20 lobules organized radially around the nipple. Milk drains from lactiferous ducts into lactiferous sinuses that meet at 4 to 18 perforations in the nipple, called nipple pores.

The pituitary hormone prolactin is instrumental in the establishment and maintenance of breast milk supply. Prolactin and other hormones prepare the breasts anatomically for the secretion of milk. When the infant suckles, sensory nerve fibers in the areola trigger a neuroendocrine reflex that results in milk secretion from lactocytes into the alveoli. The posterior pituitary releases oxytocin, which stimulates myoepithelial cells to squeeze milk from the alveoli so it can drain into the lactiferous ducts, collect in the lactiferous sinuses, and discharge through the nipple pores. It takes less than 1 minute from the time when an infant begins suckling (the latent period) until milk is secreted (the let-down). The prolactin-mediated synthesis of milk changes with time. Frequent milk removal by breastfeeding (or pumping) will maintain high circulating prolactin levels for several months. However, even with continued breastfeeding, baseline prolactin will decrease over time to its pre-pregnancy level. In addition to prolactin and oxytocin, growth hormone, cortisol, parathyroid hormone, and insulin contribute to lactation, in part by facilitating the transport of maternal amino acids, fatty acids, glucose, and calcium to breast milk.

### Pulsatile Flow Culture of Mammary Cell Types for Milk Secretion

The inventive system is designed to simulate the native environment of mammary alveoli and ducts for healthy epithelial cells (ECs) and optimal milk production by inducing variable shear stresses on the cells.

Referring to FIGS. 1A-1E, embodiments of the inventive bioreactor are designed to mirror the native environment of mammary alveoli and ducts for healthy epithelial cells (ECs) and optimal milk production. FIG. 1A provides diagrammatic side (left) and cross-sectional end (right) views showing the basic components of an exemplary embodiment of a bioreactor flow system 100, which is configured to implement adaptable shear stress within an enclosed sterile system. The incubator 102 includes a fluid-tight housing or shell 120 that encloses one or more semipermeable hollow tube 104, a pump system 106, valves 108 for controlling extracellular media fluid flow, reservoirs 110, 112, and a feedback loop 118, with valve 116, for recirculating partially processed fluid (accumulation medium) through the reactor tubing for further processing/enrichment. Shell 120 may be formed from polycarbonate or similar polymer, glass, stainless steel, or any material considered suitable for food handling, i.e., sterilizable. Fluid extracellular medium (ECM) is introduced into and circulated through the bioreactor via reservoir 110, valves 108, and inlet-outlet pipes 128 and 130. (A pump for controlling this ECM loop is not shown.) Flow through tube 104 is configured to apply mechanical shear stress to the cells within the tube as the lactating/accumulation medium flows under pressure from the pump. System controller 122 provides control signals to pump 106 to perform various operations including draining the system, e.g., applying pulsatile and/or continuous flow through tubes 104 and harvesting the milk. System controller 122 may also be configured to generate control signals to valves 108, 116, the ECM loop operation, and any additional valves and other operations within the system. In some embodiments, system controller 122 may include one or more computer processor and memory combination(s) for retaining and executing programming for automated operation of the system. In some embodiments, parameters for optimized operation of the system may be established through the use of learning machines within the system controller, with input including system sensor signals (flow rate, pressure, temperature, etc.) and quality control measurements obtained through testing of the milk product(s).

In some embodiments, pump 106 is controlled to generate continuous, unidirectional, oscillating, and pulsatile flow with adaptable flow rates to expose the cells in the tube to variable shear stresses. Generally, the range of shear stresses produced by pump 106 will be alternated among different settings selected within a range of about 2 dyn/cm² (considered “low”) and 75 dyn/cm² (considered “high”) through signals from system controller 122. Pump sequences generated by controller 122 may cause the pump flow to induce shear stresses alternating between different levels, e.g., low (within range ~2-10 dyn/cm²) to medium (range ~11-40 dyn/cm²), high (range ~41-75 dyn/cm²) to medium, high to low, or high to low to medium and back, etc. It is the variation of shear stresses on the cells via the pulsatile/oscillating flow that helps stimulate lactation. As the lactating medium flows past the cells it accumulates milk components to become the “accumulation medium”, which is processed through hollow tube 104, to be collected downstream at reservoir 112. One or more access port (not shown) may be included at or near outlet 162 to allow the accumulation medium to be sampled to determine whether it has attained the desired qualities, e.g., density, appearance, consistency, fat content, chemical content, etc. for completed milk product. If testing indicates the product is complete, the milk may be harvested. If not, the accumulation medium may be returned via valve 116 and feedback loop 118 to reactor inlet 160 for further enrichment. Alternatively, testing may not need to be performed on a regular basis once an optimal processing duration has been determined for given operating conditions.

FIG. 1B diagrammatically illustrates additional details of the inventive bioreactor structure and function, showing a section of an exemplary hollow tube 104, which, in some embodiments, may be one of a plurality of elongated tubes or lumens arranged in a parallel array extending longitudinally within a generally cylindrical outer shell 120 to collectively define an enclosed sterile bioreactor cartridge 102. The interior of tube 104 is referred to as the intracapillary or InC space 134 while the space outside of the tube (within shell 120) is referred to as the extracapillary or ExC space 132. In some embodiments, the inner surface of tube 104 may further be lined with a collagen membrane (see, e.g., FIG. 1C). Feeder (support) cells 138 and mEP cells 140 are coated onto the inner surface of hollow tube 104. It should be noted that, for convenience, primary cilia 142, which play an important role in milk production, are illustrated only for a small number of cells 140. As will be recognized by those in the art, healthy ECs should all have primary cilia. The mechanosensation resulting from the adaptable flow within tube 104 helps to keep the cells differentiated and healthy. Tube 104 may be formed from a semipermeable membrane material, e.g., polymer or similar material, through which nutrients contained within the ECM can be diffused to provide nutrition to the cells. The flow inlet for tube 104 (at the downstream end) is connected to pump system 106. In a small scale prototype implementation of the inventive bioreactor, an Ibidi™ peristaltic pump (ibidi GmbH, Graefelfing, DE) was used to provide the needed mechano-stimulation for milk production via pulsatile flow. For larger systems, one or more larger capacity pump is used to achieve the desired pulsatile flow. Milk can be harvested daily, as a batch, from the reperfusion reservoirs 112 located at the downstream end of the reactor. An optional filter 114 may be included, either at the upstream or downstream side of reservoir 112, to remove built up dead cells and other particulate materials that may be in the milk prior to harvesting.

While hollow tube 104 is described herein as “semi-permeable”, its construction is not limited to a semi-permeable material. Rather, the only requirement is that parts of the tube have sufficient permeability to allow an amount of extracellular medium into the InC to nourish the lactating cells. Thus, hollow tube 104 is still considered “semi-permeable” as a hybrid assembly of multiple sections, some of which are permeable or semi-permeable, and others of which are non-permeable. For example, a ceramic filter membrane may be used for sections of the tube to supply the cells with ECM while the remainder of the tube may be less-permeable or non-permeable. This hybrid approach allows custom control over how much ECM is available to the cells, which avoids providing too much ECM, which could potentially spoil the final product.

The embodiment illustrated in FIG. 1B includes an optional implementation in which additional mechanical stimulation is provided in the form of an assembly of opposing cylindrical rollers 150 that run along tracks 152 positioned within shell 120 so that they are parallel to the tube. The rollers slightly compress the tube from multiple sides to gently “squeeze” and push the contents of the tube, i.e., the lactating medium and accumulation medium with milk components 144, from near the inlet end towards the outlet end of the reactor. The pressure applied by the rollers should be uniform and sufficiently strong to move the fluid but gentle enough to avoid damaging the cells or dislodging them from the inner surface of the tube. Varying the speed of the rollers provides an additional means for applying adjustable shear stress. Once the rollers have completed their travel along the track, they will then be shifted apart to release pressure on the tube and moved back on the track to the starting position near the inlet end to repeat the pushing sequence. While two opposing rollers are shown, it will be readily apparent that different combinations of rails and rollers, or annular sphincters, may be used to achieve the desired inlet-to-outlet pushing action, which assists in freeing up the cells for continued milk production. Signals for control of the optional mechanical stimulation assembly will be provided by system controller 122.

FIG. 1C provides diagrammatic cross-sectional views of monolayers of lactating cells attached to the inner surfaces of each hollow fiber 104 within an array of fibers of a reactor cartridge 102. In the illustrated embodiment, the bioreactor is a modified re-perfusion reactor with multiple hollow fiber elements 104 fed by a peristaltic pump (not shown) to generate continuous, unidirectional, oscillating, and pulsatile flow with adaptable flow rates. Reactor cartridge 102 may include multi-tube bioreactors with inlets and outlets for inputting/outputting fluids to/from the intercapillary and extracapillary spaces. As illustrated, InC space inlet 160 feeds hollow tubes 104 which then output the milk (i.e., accumulation medium) to the outlet from InC space 162. Extracellular media (ECM) is fed (from reservoir 110) into ExC space via inlet 128 and then circulated back into reservoir 110 through outlet 130. The flow inlet 160 of the hollow fiber is connected to a pump for flow stimulation via pulsatile flow. In the upper left panel of the figure, transverse cross-sections of five individual hollow fibers 104 are shown with the inner surface of the outer fiber wall lined or coated with a collagen membrane 156. Cells 140 (and feeder (support) cells) are grown on the inner lumen of the hollow fiber. The collagen membrane supports a monolayer of cells so that the milk can flow through the spacing (lumen) past the cell layer. The upper right panel shows a longitudinal cross-section of a single fiber, again with the collagen membrane “liner” and the monolayer of cells 140 with the milk 170 flowing through the lumen. In this example, the inner diameter of the fiber is indicated to be 2 mm, however, as will be apparent to those in the art, the inner diameter of the fiber may be selected to support the appropriate flow parameters for generating the shear forces needed for mechanical stimulation of the cells on the inner surface of the tube.

The above-described implementation of the inventive bioreactor employs a peristaltic pump for the purpose of inducing a shear stress on the lactating cells with a continuous fluid flow within a sterile reactor environment. As will be recognized by those of skill in the art, alternative approaches for inducing controlled shear stress with continuous or variable fluid flow within a sterile environment include use of a spinning vortex, a magnetic vortex, e.g., a stirring bar, and a wave pump or valve pump. Such approaches will involve placing the vortex and/or pump within a sterile enclosure, which could negatively impact the scalability of the process relative to the above-described implementation.

As will be recognized by those of skill in the art, the number of hollow tubes within a bioreactor assembly may vary and the illustrated examples are not intended to be limiting. One example of a variation in the tube configuration is provided in FIG. 1E, where a single long tube 304 is arranged within shell 320 in a zig-zag, looped, coiled, or meandering pattern. The general direction of flow is still parallel with the length of the shell, i.e., from inlet to outlet, and may be recirculated through a feedback loop similar to those in other embodiments. In this example, tube 304 is a hybrid assembly formed from multiple tubular sections of different materials, where tube sections 344 are a permeable or semi-permeable material to allow perfusion of the ECM into the interior of the tube through these sections only. Tube sections 346 are non-permeable. Pump 306 controls the pulsatile flow through the tube from inlet to outlet. It should be noted that this is an illustrative example only and is not intended to be limiting.

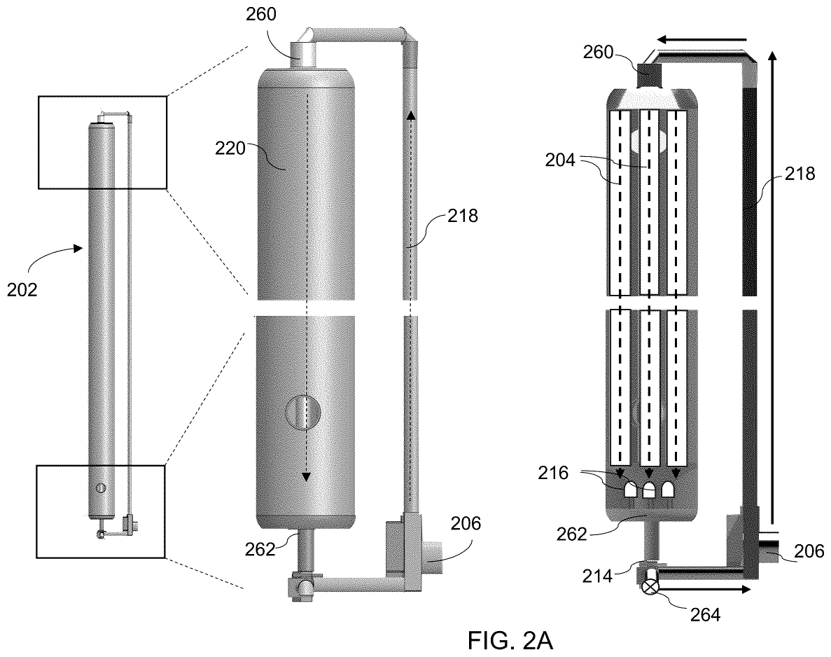

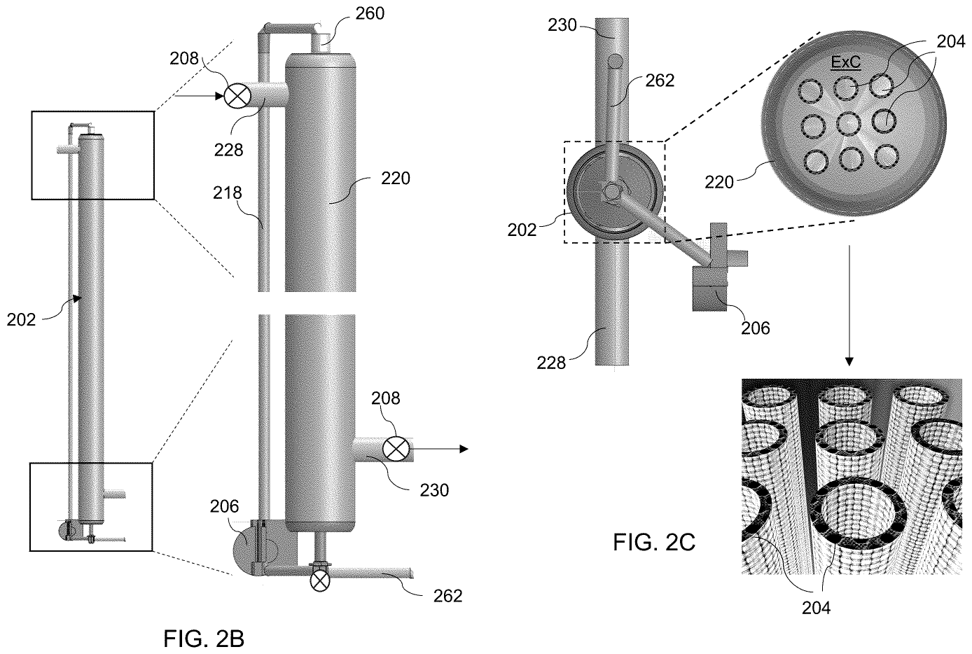

The bioreactor embodiments illustrated in FIGS. 1A-1C and described above are shown in a horizontal orientation, however, there is no requirement that any specific orientation needs to be employed. Referring to FIGS. 2A-2C, some embodiments of the bioreactor employ a vertical orientation, which may assist in scaling up the capacity of a processing facility to generate larger volumes of milk. Each cartridge 202 includes the same basic components as described above: a shell 220 enclosing one or more hollow tube reactor 204. Pump 206 feeds fluid, e.g., PBS, into the top of cartridge 202 at inlet 260 which feeds the fluid into tubes 204. Hollow tubes 204 have a monolayer of cells, specifically, ECs, coated on their inner surfaces, as shown in FIG. 2C. As in the previously-described embodiment, pump 206 is configured to induce an adaptable shear stress as fluid flows through the tubes to stimulate milk production by the cells. The vertical orientation of cartridges 202 provides gravitational assistance to the flow through the tubes. Pump 206 may recirculate partially processed milk (accumulation medium) through feedback loop 218 until the desired milk characteristics are achieved. While not shown, one or more test ports/valves may be provided at or near outlet 262 to allow testing of the processed milk to determine completion. A filter 214 may be located at outlet 262 to remove waste products. Valve 264 allows the completed milk product to be diverted to a reservoir for harvesting. The right panel of FIG. 2A provides a diagrammatic cross-sectional view of cartridge 202 with hollow tubes 204 defining the ExC (outside the tubes) and InC (inside the tubes) spaces as described above. The ECM is fed into the ExC space within shell 220 via inlet 228 and removed from the shell at outlet 230. Valves 208 and one or more pump (not shown) provide control for the ECM flow. Additional details of tubes 204 are shown in FIG. 2C, where the lower panel provides a exemplary 3-D perspective view of the tubes.

In some embodiments of the inventive bioreactor, optional light sources 216, specifically light emitting diodes (LEDs) may be included to produce different effects. In one embodiment, LEDs emitting in the blue light range, e.g., around 450 nm, have been shown to stimulate primary cilia. In a study by Prosseda, et al., blue light was used to stimulate primary ciliary regulation in cells and increase cellular contraction. (“Optogenetic stimulation of phosphoinositides reveals a critical role of primary cilia in eye pressure regulation”, Science Advances, 29 Apr. 2020, Vol. 6, Issue 18, DOI: 10.1126/sciadv.aay8699, incorporated herein by reference.) Since primary cilia of the ECs must be stimulated to produce milk, the addition of optical stimulation to the mechanical stimulation provided by the adaptable shear stress serves to increase the overall stimulation required to milk production. The optical stimulation may be further use intensity pulsing to create additional variable stimulation of the cells. Since the light is used to stimulate the ECs, the material of which tubes 204 are formed should be capable of transmission of the desired wavelength. For example, glass or clear tube sections may be used in close proximity to the LEDs. While the LEDs are shown in the lower portion of the cartridge, it will be readily apparent that they may be positioned at multiple locations throughout the interior of shell 220 to increase exposure of the ECs to the light.

In other embodiments, other light sources may be included in the cartridge or in the plumbing outside of the cartridge. For example, UV light sources (100-280 nm) may be used to enhance sterility of the product, however, care must be taken to avoid damage to the cells. Other light sources may be used to promote the health of the ECs. For example, lights sources that emit at wavelengths including 660, 700, 810 and 850 nm have been reported as promoting cell activity and growth. Control signals for operation of a light stimulation system may be provided by a system controller.

FIG. 2D diagrammatically illustrates a possible arrangement of a bank of multiple bioreactors within a production facility. Multiple such banks with any number of units, i.e., cartridges and associated plumbing, can be mounted in support frames (not shown) within the facility which may preferably include a clean room. In one possible implementation, each bank may have its own dedicated reservoir 210 for providing extracellular media to the bioreactor units within the bank and a milk collection reservoir 212 that will collect the milk produced by units within the bank. In other implementations, multiple banks may receive media from one or more larger central reservoir and output milk to one or more large central tank. Various combinations of central and dedicated reservoirs may be used. To provide an illustrative example, in a large production facility, one or more banks of bioreactor units may be used to produce different types of milk, e.g., full fat, low fat, hypoallergenic, lactose-free, or any of a variety of “designer” milk products such as those described below.

The mammary epithelium consists of two differentiated cell types organized into two cell layers, an inner layer of luminal epithelial and an outer layer of myoepithelial cells in direct contact with the basement membrane. Mammary epithelial cells (MECs) can proliferate as monolayers on plastic. However, only when receiving signals from ECM proteins plus hormones (prolactin, growth factors), i.e., structures similar to those observed in vivo, and tissue-specific gene expression (e.g., casein genes), does milk production actually occur. In some implementations, the MECs can be harvested from fresh milk as shown in one of the examples.

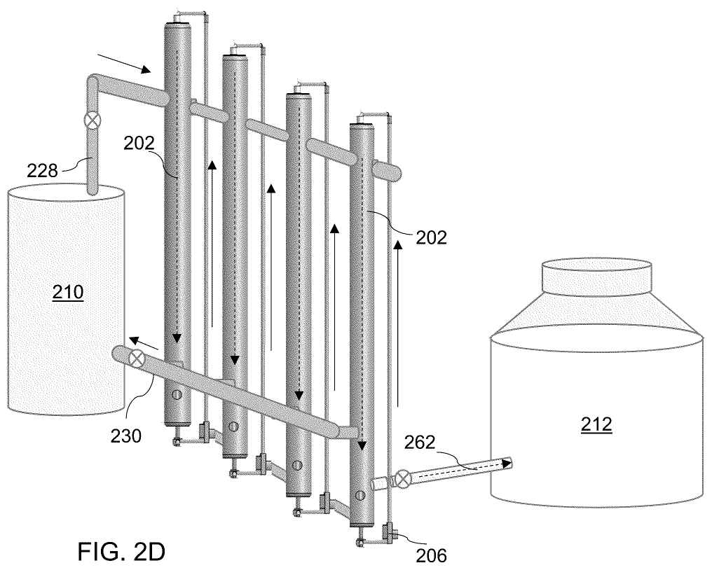

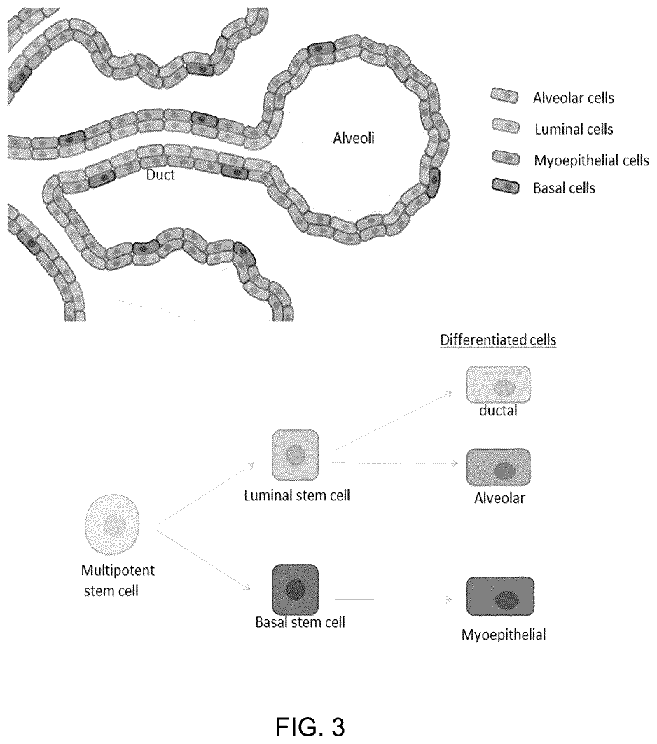

Using the disclosure herein, those of ordinary skill in the art will be able to implement similar strategies to extract MECs from the fresh milk or mammary glands using other mammary epithelial cell biomarkers. Table 2 below provides a list of biomarkers in breast tissue (see, e.g., FIG. 3) that may be useful for extraction of MECs for use in the inventive method. An exemplary selection panel may include a combination of these biomarkers. For example, a healthy breast cell panel may be assembled from a combination of biomarkers from one or a combination of sources, e.g., all, all epithelium, luminal, etc. selected from those listed in the lower seven rows of the table.

TABLE 2

| SOURCE | BIOMARKER(S) |
|---|---|
| Human mammary | MUC1, EpCAM, CD49f, CD24, CD29, CD133, Thy1, CD10, ALDH |
| epithelial cells | |
| Mouse mammary | CD24, CD29, CD49f, EpCAM, CD49b, Sca1, Prominin-1, CD61 |
| epithelial cell | |
| Bovine mammary | CD49fhigh, CD24−, CD10, KRT14, vimentin, PROCR (Basal), |
| epithelial cell | CD49flow (luminal: CD24neg [ductal], CD24+ [alveolar]), mammary |
| | stem cells: CD49fhighCD24+ (CD10, KRT14, KRT7 subpopulation with |
| | unknown function) |
| Basal/myoepithelial | CD44, CD90, CD49f, Cytokeratin 14, CD10, SMA, Cytokeratin 5, |
| mammary epithelial cell | Vimentin, CD271, Connexin 43, Integrin α9β1, p63, P-Cadherin |
| Luminal mammary | EpCAM+, CD49flo (non-clonogenic), CD49f, EpCAM/CD326, |
| epithelial cell | Cytokeratin 7, Cytokeratin 8, Cytokeratin 10, Cytokeratin 18, |
| | Cytokeratin 19, Muc1/CD227, CD24, CD133/PROM1, Her2, CD117, |
| | CD166, CD282, CD340, Claudin 4, C-MET, ER, PR, VDR |
| Luminal (clonogenic) | EpCAM+, CD49fhi, ALDH+, |
| mammary epithelial cell | |
| Further breast cells | BRCA1, CD49b, CD66, EGFR, CD321 |
| (healthy tissue) | |
| Stromal - absent in | CD26, CD34, CD39, CD45, CD61, CD73, CD140b, HPC |
| epithelial cells (negative | |
| selection) | |
| Variable expression | Erα, PR, SMA, EGFR, CD90, CD10, CD44, CD54/I-CAM |
| (healthy tissue) | |

Healthy breast cell panel

| | |
|---|---|
| All breast cells | CD9, CD47, CD54, CD59, CD95, CD164 |
| All epithelium | CD49b, CD66 |
| Luminal | CD24, CK8/18, CK19, MUC1, GATA3, EPCAM |
| Luminal progenitor | CD13, CD15s/CD73/CLA |
| Mature luminal | +CD166/CD227/CD340 |
| Myoepithelial | CD10, CD90, CD200, CD29/CD142/CD271, CK5, CK14, CK17, SMA, |
| | V1M |
| Stromal | CD34/CD39/CD140b, CD49e |

Pulsatile Flow: Pulsatile flow is a periodic pressure fluctuation wave traveling along the flow path such as that created in the hollow tube(s) of the bioreactor. Pulsatile flow systems can closely mimic blood flow characteristics within the heart and vasculature system.

The Mammary Endothelial cells (MEC) adapt their morphology and function to the in vivo hemodynamic environment in which they reside. In vitro experiments indicate that similar alterations occur for cultured MEC exposed to a laminar steady-state flow-induced shear stress. However, in vivo MEC are exposed to a pulsatile flow environment; thus, in this investigation, the influence of pulsatile flow on cell shape and orientation and on actin microfilament localization in confluent bovine aortic endothelial cell (BAEC). These results demonstrate that EC can discriminate between different types of pulsatile flow environments. Furthermore, these experiments indicate the importance of engineering the cell culture environment so as to include pulsatile flow in investigations of endothelial cell biology.

Elimination of FCS from the Lactating Medium: Today, standard culture media rely on established yet outdated notions that blood serum from unborn calves (FCS) is required, which has served as the “gold standard” since the 1960s. For many years, problems with FCS, and the reasons it needs to be replaced with better alternatives, have become an area of considerable research interest. These problems with FCS include the fact that: (a) its production is a cruel undertaking, and (b) FCS cannot be used in clean milk production for human consumption due to reported serum contamination with viruses, along with other safety concerns in terms of endotoxins, Mycoplasma, RNA contaminants, or prion proteins. Accordingly, the medium used for culturing the cells for use in the inventive bioreactor is preferably FCS- and hormone-free (except for prolactin) as well as being based on the latest scientific research and cell culturing techniques.

Serum-starvation in general can make cells unhealthy, however the adverse effect can be counteracted by flow modulation. Changes in the flow strength can induce cilia growth. Therefore, cilia need to be stimulated using flow to achieve milk-production.

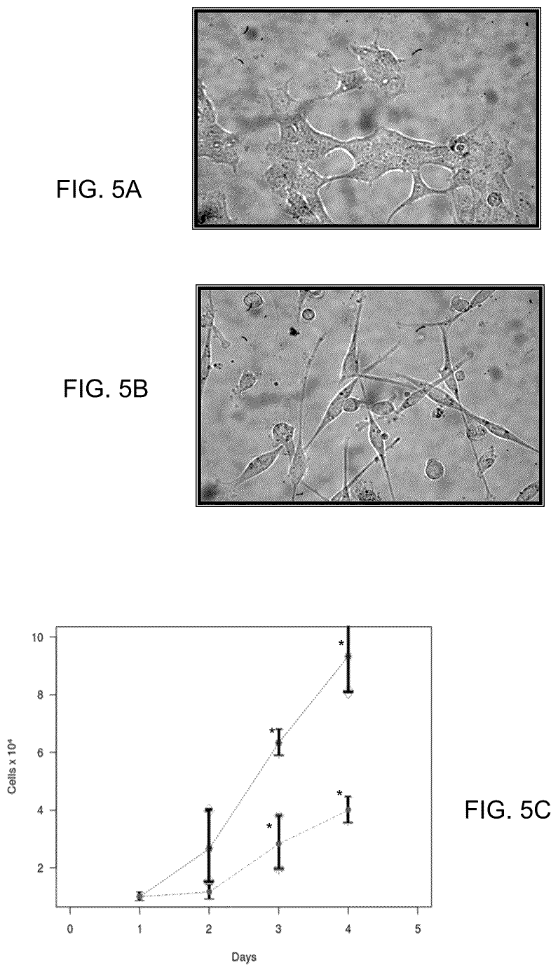

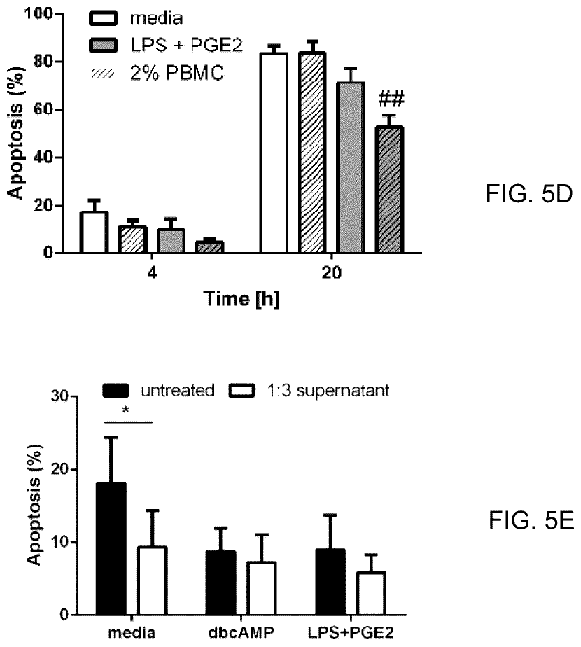

Mechanical stimulation (Flow) for healthy EC cells to counteract elimination of FCS from the lactating medium: Hormonal and mechanical signals can both induce gene expression changes in mammary epithelial cells (MEC) that culminate in milk synthesis and secretion. Using serum-free media and a combination of hormonal and mechanical stimuli, bovine mammary epithelial cells (BMEC) were transitioned from primary multicellular organoids to coordinated 3-dimensional ductal networks. See, for example, FIGS. 5A-5B showing mammary epithelial cells possessing an epithelial-like phenotype and mammary myoepithelial cells possessing a spindle-like phenotype, respectively. At the structural level there was evidence that mechanical signaling via gel release, prolactin, and the combination of the two resulted in changes in cellular morphology that were consistent with initiation of stage I lactogenesis.

Endothelial cells require mechanical stimulation to grow healthy. Studies have shown that a certain type of flow can provide shear mechanical stimulation to EC cells, resulting in increased cell performance. In some embodiments, constant mechanical stimulation for healthy ECs resulted in an increased, hormone-free milk-production process. It is important to understand the nature of the stimulation applied to the cells, the principles of which are illustrated in FIG. 4. Mechanical strain occurs when a force acts directly against the cell. With high strain, the endothelial barrier can be loosened, resulting in cell death and high cell turnover. Epigenetic modifications and inflammation can occur. In contrast, mechanical shear involves parallel movement of the fluid over top of the cells, which improves cell survival with low turnover. The goal is, thus, to avoid mechanical strain while increasing mechanical shear to stimulate cellular function. The lower panel of FIG. 4 provides a comparison of a cell layer after (left) and before (right) application of shear stress.

In some embodiments, lactating media and test cultures are fluidically driven with a peristaltic pump, for example, the Ibidi™ pump system used in a smaller scale prototype bioreactor, or other pump capable of producing pulsatile flow that provides adaptable shear stress in an enclosed sterile system. In some embodiments, mechanical shear stress is assessed and constantly monitored.

Feeder Cells: Feeder or support cells 138 comprise a layer of cells that are unable to divide, but which provide extracellular secretions to promote proliferation of other cells, i.e., lactating cells 140. Feeder cells differ from a co-culture system because only one cell type is able to proliferate.

In some embodiments, feeder cells are co-cultured with MECs and form a monolayer of lactating cells attached to the inner lumen of the elongated hollow fiber bioreactor. In some embodiments, feeder cells may be seeded in the extracapillary (ExC) space surrounding the hollow fibers.

Hollow Fiber Bioreactor: A hollow fiber bioreactor is a 3-dimensional cell culturing system based on hollow fibers, which, in some embodiments, are small, semi-permeable capillary membranes arranged in an array with a typical molecular weight cut-off (MWCO) range of 10-30 kDa. Exemplary implementations are described above with reference to FIGS. 1A-1C, 2A-2D. When multiple tubes are employed, the tubes may be bundled and housed within cylindrical shells of polycarbonate, glass, stainless steel, or other appropriate material, i.e., sterilizable, to create hollow fiber bioreactor cartridges. Within the cartridges, which are also fitted with inlet and outlet ports, are two compartments: the intracapillary (InC) space 134 within the hollow fibers, and the extracapillary (ExC) space 132 surrounding the hollow fibers.

Cells are seeded into InC space 134 (FIG. 1B) of the hollow fiber bioreactor 104 and expand therein to coat the inner surface of the hollow fiber 104 with a monolayer. Cell culture medium is pumped through ExC space 132 to deliver oxygen and nutrients to the cells via hollow fiber membrane perfusion. As the cells expand, their waste products and CO₂ also perfuse the hollow fiber membranes and are carried away by the pumping of the milk accumulation medium through InC space 134. As waste products build up due to increased cell mass within the InC, the rate of medium flow may need to be increased so that cell growth is not inhibited by waste product toxicity. Waste products can be filtered out by a filter disposed within or near the reservoir after the accumulation medium has been fully enriched, i.e., milk production has been completed.

Supporting Extracellular Matrix: The mammary epithelium may include two differentiated cell types organized into two cell layers, an inner layer of luminal epithelial and an outer layer of myoepithelial cells in direct contact with the basement membrane. Mammary epithelial cells can proliferate as monolayers on plastic. However, only when receiving signals from ECM proteins plus hormones (prolactin, growth factors), structures similar to those observed in vivo, and tissue-specific gene expression (e.g., casein genes), does milk production occur. Thus, in order to simulate natural milk production, the bioreactor must re-create these structures and an environment with the same (or similar) stimuli as occur in an in vivo setting. In some embodiments, this can be achieved by adjusting the supporting extracellular matrix, the composition of culture media and by the co-culture of necessary cell types to achieve functional differentiation.

Other successful approaches may also stimulate milk genes. In some embodiments, cells were shown to reorganize and form 3D structures like those observed in vivo and exhibiting milk protein genes expression on collagen gels. In some embodiments, epithelial cells grown in laminin-rich gels underwent functional differentiation based on the expression of the casein genes. Natural and synthetic polymeric materials have been investigated as alternatives to ECM proteins.

In some embodiments, 3D bioprinting of mammary tissue may be used. In some embodiments, 3D culture procedures including lactogenic hormones (prolactin, insulin and hydrocortisone) are able to induce caseins synthesis from primary mouse epithelial cells embedded in floating collagen gels. In some embodiments, differentiation medium, complemented with oleic acid, pituitary extract, and dexamethasone, was shown to induce the synthesis of major milk constituents such as β-casein, triglycerides and lactose, in a 3D in vitro model of bovine primary mammary epithelial cells grown on a cell culture insert coated with collagen.

Morphological and functional differentiation of cryopreserved lactating bovine mammary cells can be cultured on floating collagen gels. In some embodiments, bovine primary epithelial cells from lactating mammary tissue grown on floating collagen gel exhibited a polarized conformation with high differentiation status evidenced by the observation of apical microvilli, tight junctions, and fat droplets surrounded by casein-containing secretory vesicles.

The use of collagen gel provides the advantage of mimicking in vivo conditions and allows relatively long-term studies.

The milk produced by the inventive bioreactor has the ability to match nutritional content, taste, and quality of milk obtained traditionally.

### EXAMPLES

Aspects of the inventive system and method may be further understood in light of the following examples, which should not be construed as limiting the scope of the present teachings in any way.

### Example 1: Bioreactor

As described above, the inventive bioreactor is designed to mirror the native environment of mammary alveoli and ducts for healthy epithelial cells (ECs) and optimal milk production. The goal of the system and method is to employ process control conditions and cell biology to provide an FCS-free, or substantially FCS-free, culture of primary ECs. In some embodiments, constant mechanical stimulation for healthy ECs results in an increased, hormone-free milk-production process. (Noting that the hormone prolactin is still necessary.) In some embodiments, lactating media and test cultures are fluidically driven with a peristaltic pump to produce adaptable shear stress in an enclosed sterile system. In some embodiments, mechanical shear stress may be assessed and continuously or periodically monitored. In some embodiments, the bioreactor is a modified re-perfusion reactor with a hollow fiber module, controlled by peristaltic pump to generate continuous unidirectional, oscillating, and pulsatile flow with adaptable flow rates. In some embodiments, lactating cells are grown or coated on the inner surface of the hollow tube 104. The inlet of each hollow tube is connected to the pump to provide flow stimulation via pulsatile flow. Milk may be harvested in batches at regular intervals, from periods ranging from daily to weekly to monthly, depending on the scale (volume) of the overall bioreactor system. The system supplies an extracellular medium through permeable and/or semipermeable membranes in the tube to nourish the lactating cells inside the tube. The ECM may be replenished and/or replaced periodically through the processing period.

### Example 2: Mammary Epithelial Cells (MECs) Harvest

Fresh bovine milk (i.e., from Guernsey or Galloway cows) was defatted by low-speed centrifugation at typical refrigeration temperature (~4 °C). The skim milk was removed, and the remaining total cell pellet was washed multiple times in phosphate buffered saline (PBS) and resuspended in PBS. Immunomagnetic separation was used to isolate the MECs from the total milk somatic cells and to remove the leukocytes, which were collected separately for further use. Immunomagnetic separation involved incubating the total milk cell suspension with magnetic beads (Dynabeads) coated with a primary monoclonal antibody directed against cytokeratin 8 (K8.13) (Boutinaud et al., 2008). The resulting antibody-bound MECs were then washed and the purified MECs were resuspended in PBS. MECs were then cultured in complete medium for 1 week to ensure sufficient cellular proliferation. The complete medium included DMEM/F12 (alternatively EpiCult) supplemented with 2% FBS or FCS-replacement, 5 μg/ml insulin, 100 ng/ml hydrocortisone L-Glutamine, optionally supplemented with antibiotic.

### Example 3: MEC Culture & Lactation

Seeding of mammary myoepithelial and epithelial cells (MEPs) in the complete medium was performed and maintained until a monolayer was established. Low shear stress was used for in-flow in complete medium for cellular proliferation (1 week). Shear stress was 0.007 dyn/cm². Next, a sedimentation and attachment period was carried out in the hollow fiber reactor for ~4 hours. Physiological shear-stress at about 5 dyn/cm², i.e., low shear stress, was performed for proliferation and monolayer formation over a period of several days. The media was changed every 3 days or fed-batch culture.

Next, serum withdrawal and supplementation with lactation media was performed to induce lactogenic differentiation. Pulsatile flow was initiated, with the pump being controlled to periodically change fluid flow to induce variable shear stresses alternating at different levels within a range from around 2 to 75 dyn/cm² over a period of at least one week, more preferably about 2-3 weeks. The total culturing time will vary depending on system size (volume), with a small scale system taking around 2 weeks and larger scale systems taking up to multiple months for recirculating the accumulation medium through the reactor for complete culturing. Over a multi-week period, lactation media changes were made every 3 days, or fed-batch culture. The product was then harvested after culturing was complete. Processing after harvest included preservation, by one or more of pasteurization, freezing, or dehydration. Quality control testing included evaluation of bacterial, Mycoplasma, fungi, and principal components.

### Example 4: Using Matrix to Replace Feeder Cells

Feeder cells are generally suitable for supporting stem cell co-culturing, however, when dealing with different species, this could create a potential for transfer of animal pathogens and/or unwanted immune response that could impact milk production. Matrigel matrix structures offer a suitable and functional support structure to cells requiring feeder cells.

For coating of Matrigel (BD Biosciences, Bedford, MA), 500 μL of BD Matrigel was transferred into each well of six wells plate on ice and spread by rocking gently. The plates were then incubated at 37 °C for 2 h to allow the BD Matrigel to polymerize into gel. Then, the suspensions of reprogrammed HDFs (human dermal fibroblasts) (2.4×10⁴/cm²) were transferred onto coated palates with BD Matrigel matrix in DMEM media containing 10% FBS and the plate was returned into incubator. After 48 h the media was replaced by supplemented DMEM/F-12 (including supplemented factors as mentioned above) and was changed every day for formation of hiPSC colonies. The iPSCs culture was maintained and followed for 10 passages.

### Example 5: Preparation of Feeder Cells with Conventional Method

CF-1 MEFs of passage 3 (P3) at 80-90% confluence were inactivated with 10 μg/ml of MMC (Hisun Pharmaceutical Company, China) for 0, 0.5, 1.0, 1.5, and 2.0 h at 37 °C. After the incubations, the cells were washed with PBS 6 times, trypsinized, centrifuged at 180×g for 5 min, and re-suspended in MEF medium. Cells were counted and frozen for later use.

### Example 6: Preparation of Feeder Cells with Suspension-Adhesion Method

We prepared feeder cells by SAM according to FIG. 1. Briefly, CF-1 MEFs of P3 were cultured for four days, digested to single cells with 0.25% trypsin/EDTA (Dalian Meilun Biotech Co., Ltd, China), and collected in 50 ml-centrifuge tubes. The cells were seeded at 8×104-1.1×105 cells/cm² in 10 cm-dishes. MMC (10 μg/ml) was added after 2.0-3.0 h at 37 °C. Medium containing MMC was discarded 0.5-3.5 h post-treatment. The cells were then washed with PBS 6 times, trypsinized, centrifuged at 180×g for 5 min, and resuspended in MEF medium. Cells were counted and frozen for later use.

### Example 7: Preparation of Feeder Cells with Three-Dimensional (3D) Suspension Method

After connecting the CELLSPIN System (5-75 RPM, CELLSPIN System with glass-ball stirring pendulum, Integra Bio-Sciences, Switzerland) to the incubator, spinner flasks were sterilized by autoclaving. Feeder cells were prepared by 3DSM. Briefly, CF-1 MEFs of P3 growing for four days were digested to single cells by 0.25% trypsin/EDTA and collected into a 50 ml-centrifuge tube. Cells were transferred to spinner flasks with glass ball pendulum, which accommodate 25-1000 ml of volume, at a density of 0.5-1.3×106 cells/ml. MMC were added at 10 μg/ml. After incubation for 0.5, 1.0, 1.5, and 2.0 h at 37 °C, the cells were centrifuged at 180×g for 5 min, washed with PBS 3 times, resuspended in MEF medium, counted, and cryopreserved for later use.

### Example 8: Production Feeder Cells/or Support Matrix for Bioreactor

Bovine embryonic stem cells (ESCs) are powerful tools for agricultural and biomedical applications and have been successfully cultured and differentiated using supporting feeder cells (Cell Reprogram. 2012 December; 14 (6): 520-9. doi: 10.1089/cell.2012.0038). Currently, commercially available feeder cells are exclusively mouse or human-derived feeder cells. One of the goals of the inventive system and method is to produce custom feeder cells derived from cow fibroblasts. Potential feeder cells that may be used in embodiments of the inventive method include fibroblasts, mEP and myoepithelial cells, and mEP on fibrous extracellular matrix.

Alternative synthetic methods may be employed as support for milk producing cells. The following examples identify a few of these methods.

### Example 9: Lab-Grown FCS Alternative

Upon activation, PBMCs secrete a powerful cocktail of pro-survival factors that helps them to combat infections. These are secreted into the blood and become part of the serum-phase. Co-culture experiments show that the supplementation of cells with the supernatant phase of cultivated PBMCs significantly prolongs their survival. In cell culture, this is used by the “tight packing” of cells to rely on the microenvironmental factors, i.e., suspensions at a high cell density to achieve healthy cell growth and recovery after freezing. Addition of Innate Immune cell supernatant provides a survival-factor, as strong as a comparable direct survival stimulation.

PBMCs were extracted from the peripheral blood of volunteers using Percoll gradients. After supplying them with a pro-activation stimulus, containing A) LPS B) opsonized beads C) Mechanical stimulation in an Ibidi™ pump system, the cells were maintained in a stimulated state and the supernatant was enriched for 24 to 48 hours and then collected. The supernatant phase was separated from the cellular fraction and used for the supplementation of media. For future up-scaling, conditionally immortalized ER-Hoxb8 hematopoietic progenitor cells can be used and differentiated on demand into PBMCs, to provide an infinite supply of PBMCs.

### Example 10: EpiCult™ Plus Mammary Epithelial Serum-Free Medium

Successful tests in mammary cells were performed using cell culturing techniques adapted from custom modifications of cell culturing methods of Epithelial cells (Mammary) from FCS-free Database (https://fcs-free.org/fcs-database) Animal free research Foundation.

FIGS. 5A-5E provide representative growth curves & images. Photographic images of mammary breast tissue cells were taken using live-cell microscopy with a Kodak Imaging System, where FIG. 5A shows mammary epithelial cells, possessing an epithelial-like phenotype and FIG. 5B shows mammary myoepithelial cells, possessing a spindle-like phenotype. FIG. 5C provides growth rate curves of breast tissue cell lines. 1×10⁴ cells were seeded and measurements were subsequently taken on the 4 following days. Values for three independent cell samples for each day were compared by using unpaired t-tests. Significance between mammary epithelial cells (green) and mammary myoepithelial cells (red) cell growth rates was thereby determined for day 3 (p=0.007) and day 4 (p=0.0228). These values were highlighted in the diagram through asterisks. FIGS. 5D-5E are plots showing that addition of 2% PBMCs (5D) or 1:3 neutrophil conditioned media (i.e., supernatant) (5E) provides a survival stimulus comparable to the addition of pro-survival mediators dbcAMP or LPS and PGE2 in cultured immune cells.

In some implementations, a learning machine (AI)-guided approach may be used to optimize media composition and as well as optimizing and adjusting processing parameters.

### Example 11: In Vitro Milk Production Using Extracted Mammary Epithelial Progenitors from Fresh Bovine Milk

The bioreactor was seeded with mammary epithelial cells (MEPs) in complete medium supplemented with 2% FCS (FCS replacement), 5 μg/ml insulin, 100 ng/ml hydrocortisone L-Glutamine, optionally supplemented with antibiotic, IGF-I, or EGF. The complete medium can be selected from DMEM, F12 or EpiCult. MEPs were incubated until a monolayer was established. A low shear rate was applied for in-flow in complete medium to maintain cellular proliferation for 1 week. The sedimentation and attachment period in hollow fiber reactor was 4 hours. Medium to high shear-stress was maintained for proliferation and monolayer formation for 2 days. Full media changes were performed every 3 days or fed-batch cultured. Serum withdrawal and supplementation with lactation media was performed to induce lactogenic differentiation. The lactation medium included DMEM/F12 (alternatively EpiCult) supplemented with 2% FBS (or FCS-placement), L-Glutamine, 1-5 μg/ml prolactin (or shear stress as defined above). Pulsatile flow was induced by alternately switching the pump between different pump rates, e.g., high, medium, and/or low, in order to create shear stresses within a range of 2 to 75 dyn/cm². This step was continued for a period of at least one week, more preferably about 2-3 weeks. As will be apparent to those in the art, the total processing time will depend on system size (volume). A small scale system may take as few as 2 weeks to produce fully cultured milk, while larger scale systems could take up to a few months for complete culturing. Lactation medium changes were performed every 3 days or fed-batch culture. The product was then harvested and processed as described in Example 3.

### Example 12: Genetic Engineering to Induce Hyperlactation and Other Modifications

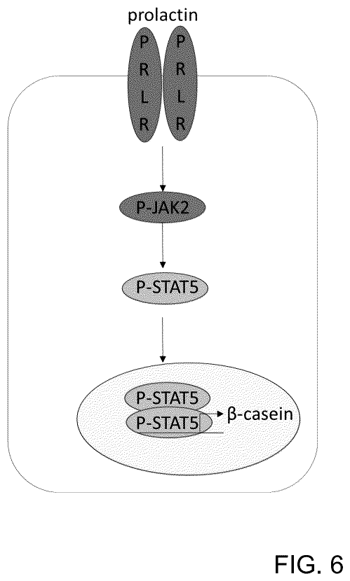

Hyperlactation can be induced in cells using CRISPR technology. Referring to the diagram in FIG. 6, the following protocol may be used: CSN2 polymorphisms (SNPs): His220, G->A in Glu223; LALBA Polymorphism: in the 5′LALBA promoter; PRLR SNPs: rs62355518, rs10941235, rs1610218, rs34024951, rs9292575; PRL SNPs: rs849872; Jak2 gain of function mutation for hyperactivation: V617F. Additionally, pharmaceutical interventions may include use of Retinoid acid and ATRA to target CSN3 expression.

SNPs can be modified (via CRISPR) to decrease lactose content for diet, lactose-free milk: CSN2 (Glu340, Thr174, Lys14), B4GALT1 (T224A).

Hypoallergenic milk can be produced using a CRISPR cluster bomb to introduce point mutations in the base pairs 186 and 213 of the gene CSN1S1, corresponding to the sites CDS 84-123 (CSN1S1) and CDS 87-123 (CSN1S2).

Table 3 below lists a number of potential modifications that can be achieved through genetic engineering of mEPs and mEP-derived cell lines.

TABLE 3

| Gene | Polymorphisms | Location | Result |
|---|---|---|---|
| CSN2 | Point mutations, SNPs | His220, G->A in | Hyperlactation |
| | | Glu223 | |
| CSN2 | Point mutations, SNPs | Glu340, Thr174, | decreased lactose |
| | | Lys14 | content for diet, |
| | | | lactose-free milk |
| CSN1S1 | Point mutations; SNPs | bp186 and 213; | Hypoallergenic |
| | | CDS 84-123 | |
| CSN1S2 | Point mutations; SNPs | CDS 87-123 | Hypoallergenic |
| CSN3 | — | Retinoid acid and | Increased CSN3 |
| | | derivatives or ATRA | content |
| | | agonists | |
| B4GALT1 | Point mutations, SNPs | T224A | decreased lactose |
| | | | content for diet, |
| | | | lactose-free milk |
| LALBA | Point mutations; SNPs | 5′LALBA promoter | Hyperlactation |
| PRLR | Point mutations, SNPs | rs62355518, | Hyperlactation |
| | | rs10941235, | |
| | | rs1610218, | |
| | | rs34024951, | |
| | | rs9292575 | |
| PRL | Point mutation; SNPs | rs849872 | Hyperlactation |
| Jak2 | Mutations | V617F | Jak2 hyperactivation; |
| | | | Hyperlactation |
| CSN1S1 | CRISPR | CSN1S2 to replace | Hypoallergenic |
| | | CSN1S1 | |

Particularly desirable modifications that are enabled by the inventive methods include the production of A2 rich milk from cells.

### Example 12: Cultivation Process

Cell Selection—Cell Type: The inventive processes utilize mammary epithelial cells specifically chosen for their high milk-producing capabilities. These cells are carefully selected and isolated from healthy donor animals or sourced from established cell lines renowned for efficient milk production. These selected cells exhibit superior productivity, allowing for enhanced milk yield and quality compared to other cell types commonly used in cellular agriculture.

Cell selection is guided by multiple considerations. The milk of each species has a unique composition that has evolved over millions of years to suit the needs of infants of that species. Milk contains a myriad of immunological, biochemical and cellular components that have the potential to significantly alter newborn immunity and susceptibility to infection. Functionally, it is possible to distinguish between nutritional and bioactive components in mother's milk. The latter are growth and immunological factors and cellular components. Typically, breast milk is thought to contain epithelial cells and immune cells. Recent breakthroughs have shown that breast milk is more heterogeneous than previously thought and that it also contains stem cells. Furthermore, breast milk is also a continuous source of commensal and beneficial bacteria, including lactic acid bacteria and bifidobacteria.

A key component of milk is immunological cells. For example, human milk-mediated protection of the infant has long been known and intensively studied. Breast milk confers active and passive immunity to the infant because it is a rich source of immunoglobulins, lactoferrin, lysozymes, cytokines, and numerous other immunological factors.

New data have revealed that leucocytes constitute only a small minority (<2%) of the cells in the mature milk of a healthy mother. Leukocytes primarily provide active immunity and promote the development of immunocompetence in the infant, but it is also probable that they protect the mammary gland against infection.

Breast milk leukocytes have been shown to be activated, motile and interactive, and they can be transferred via the systemic circulation into distant tissues. It has been postulated that miRNAs, which are abundant in breast milk, also participate in leukocyte survival in the infant's gastrointestinal tract, potentially conferring immunoprotective and developmental functions.

The stage of lactation is associated with major changes in milk leukocyte composition. Using multicolor flow cytometry to identify and quantify leukocyte subsets in breast milk obtained from healthy women, Trend et al. found that colostrum contains approximately 146,000 cells/ml and that the amount decreases in transitional (8-12 day postpartum) and mature milk (26-30 day postpartum) to 27,500 and 23,650 cells/ml, respectively]. They also demonstrated that breast milk contains a greater variety and complexity of leukocyte subsets than previously thought. Of the identified cells, the major leukocytes present were the myeloid precursors (9-20%), neutrophils (12-27%), immature granulocytes (8-17%), and non-cytotoxic T cells (6-7%). Progression of lactation is associated with decreasing major CD45+leukocyte concentration, eosinophils, myeloid and B cell precursors, and CD16-monocytes. The relative frequencies of neutrophils and immature granulocytes significantly increased in mature milk in comparison to colostrum.

A second key component of milk is non-immune cells and stem/progenitor human breast milk cells (and markers). Table 4 provides a list of somatic cells in fresh breast milk when both mother and infant are healthy.

TABLE 4

| | | % of the total cell population | |
|---|---|---|---|
| | | Colostrum (1 day prepartum | Peak lactation |
| Somatic cells | Markers | or 1 day postpartum) | (month postpartum) |
| Leukocytes | CD45 | 13-20 | 1-2 |
| Myoepithelial cells | CK5, CK14, CK18, CK19, | 50-90 | 60-98 |
| | CD49f, SMA | | |
| Lactocytes | CK18, EPCAM | | |
| Breast milk stem cells | CD44, ITGB1/CD29, | 10-15 | No data |
| (hBSCs) | ATXN1/SCA1 | | |
| Mesenchymal stem | CD90, CD105, CD73, | | |
| cells (MSCs) | VIM | | |

While the nutritional and protective function of breast milk has been previously examined, little is known about the properties and roles of the non-immune cells that are present. Studies performed in the 1950s revealed that colostrum contains epithelial cells. In the last decade, it was shown that in addition to these cell populations, breast milk contains stem and progenitor cells.

Thus, human breastmilk contains heterogeneous cell populations including lactocytes (milk-secretory cells), myoepithelial cells (from the ducts and alveoli of mammary gland) and a hierarchy of progenitor and stem cells.

Luminal and myoepithelial cells and their precursors represent nearly 98% of the non-immune cell types in human milk under healthy conditions. They express a few membrane antigens: CK5, CK14 and CK18, which are markers of differentiation of mammary epithelial cells.

Myoepithelial cells build smooth muscle fibers surrounding the alveoli. Their contraction results in the expulsion of milk from the alveoli into the milk ducts. Luminal cells express epithelial cell adhesion molecule (EPCAM), whereas myoepithelial cells express smooth muscle actin (SMA) and cytokeratin 14 (CK14). Lactocytes line the alveoli of the human mammary gland and are responsible for the synthesis and secretion of milk into the alveolar lumen. These alveolar cells express cytokeratin 18 (CK18) and synthesize milk proteins such as α-lactalbumin and ß-casein. Mammary precursors to both luminal and myoepithelial cell types express α6 integrin (CD49f) and cytokeratin 5 (CK5). Many studies demonstrate that epithelial cells isolated from fresh breast milk are adherent cells that form colonies of various morphologies that can be maintained through multiple in vitro culture passages.

A few studies have suggested that human milk contains mesenchymal stem cells (MSCs). In a study conducted in 2013, cells expressing the typical MSC markers, like CD90, CD105 and CD73, were isolated from breast milk. Subsequent studies concluded that no convincing evidence currently exists supporting the presence of MSCs in breast milk.

A third key component of milk is probiotics. Human milk is far from being a sterile fluid. The existence of the human milk microbiome was discovered only a decade ago. It is estimated that an infant feeding on 800 ml of breast milk per day could ingest 107-108 bacterial cells daily. Advances in the assessment of early host-microbe interactions suggest that early colonization of the infant gut by milk bacteria may have an impact on disease prevention in children and later health. The most frequent bacteria found in human milk are those belonging to the species Staphylococcus, Acinetobacter, Streptococcus, Pseudomonas, Lactococcus, Enterococcus and Lactobacillus. Some, like Staphylococcus, Corynebacterium or Propionibacterium, can be isolated from the skin and are also frequently found in human milk. They probably prevent from colonization of the host by more severe pathogens, such as S. aureus. Others, including L. gasseri, L. salivarius, L. rhamnosus, L. plantarum and L. fermentum, are considered probiotic species by the European Food Safety Authority (EFSA).

Culture Medium and Conditions: Nutrient-Rich Medium: The cultivation process involves the use of a proprietary nutrient-rich medium comprising a blend of growth factors, hormones, vitamins, and essential nutrients necessary for the optimal growth and milk synthesis by mammary epithelial cells.

Optimized Conditions: Cultivation occurs in bioreactors or cultivation chambers equipped with precise control systems. The environmental conditions, including temperature maintained at 37 °C, pH maintained around 7.4, and controlled oxygen levels, are meticulously regulated to mimic the physiological conditions necessary for robust cell growth and milk production.

Bioreactor Setup:—Design and Functionality: The bioreactor system consists of sterile, closed systems designed to support large-scale cell culture. These bioreactors provide a controlled environment, ensuring aseptic conditions and facilitating continuous cell growth and nutrient exchange.

Feeding Regimen: The system employs a continuous feeding regimen where the nutrient-rich medium is supplied in controlled intervals to sustain cell growth and milk synthesis throughout the cultivation period.

Harvesting Stage:—Optimal Harvesting Time: The cells are harvested at a specific stage of growth when they reach peak milk-producing capacity. This stage is determined through continuous monitoring of cellular parameters, such as lactation markers, cell density, and milk protein expression levels.

Harvesting Technique: Gentle and non-invasive techniques are employed to collect the cell-cultivated milk while preserving cell viability and ensuring minimal disruption to the cultivated cell population.

This cultivation process aims to maximize milk yield, quality, and efficiency while maintaining strict control over the growth conditions to ensure consistent and high-quality cell-cultivated milk for use in various products, including food and non-food products, as described herein.

### Example 13: Chocolate Production

Integration Method: Cell-cultivated milk (CCM) is incorporated into the chocolate production process by replacing traditional dairy milk. The process involves blending the CCM with cocoa solids, sugar, and other necessary ingredients in precise proportions. As is known in the art, typical chocolate manufacturing processes utilize milk powder formed by dehydrating liquid milk through a series of drying processes, e.g., freeze- or spray-drying, until it is powder. In some embodiments, the processing of the cell-cultivated milk used to produce milk powder can be optimized for the specific needs of the drying process, for example, to enhance the efficiency of mixing, or to confer certain desirable qualities on the chocolate, such as richness or smoothness. In general, the dehydrating process itself depends on characteristics of the milk, e.g., milk fat and solids content. Accordingly, the use of cell-cultivated milk in the creation of milk powder allows desired features of the milk powder to be engineered and/or fine-tuned within the milk cultivation process to meet specific requirements. After drying, the milk powder can be used immediately or may be stored in an airtight container in a cool, dry place, and used (for chocolate or other food production) within 1-2 months for best quality.

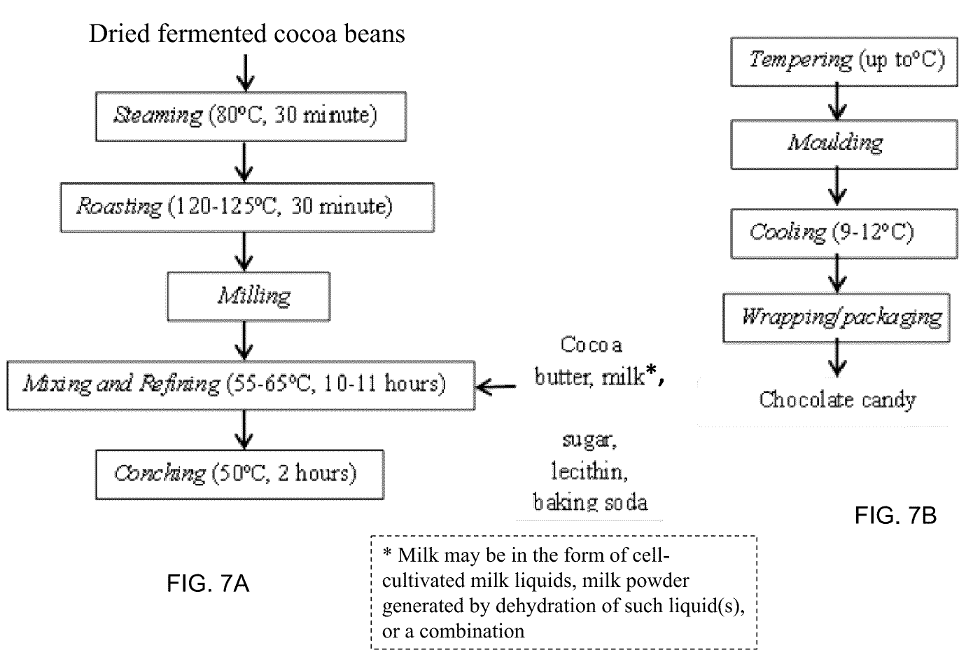

A helpful reference that explains the details and rationale for the steps of conventional chocolate processing is V. Barisic, et al., “The Chemistry behind Chocolate Production, Molecules, 2019 September; 24 (17): 3163, which is incorporated herein by reference. FIGS. 7A and 7B illustrate exemplary process flows for processing of bulk chocolate and production of chocolate candy, respectively. In FIG. 7A, the mixing and refining step involves the addition of cell-cultivated milk as described above in place of conventional cow's milk. As noted by the “*”, the milk used in the mixing step may be cell-cultivated milk in liquid form, milk powder produced by the dehydration, spray-drying or freeze-drying of cell-cultivated milk liquid, or some combination thereof.

As is known to those in the art, there are several different types of chocolate classified primarily according to the proportion of cocoa and fat content used in a particular formulation. Milk chocolate contains, on a dry matter basis, not less than 25% cocoa solids (including a minimum of 2.5% fat-free cocoa solids) and a specified minimum of milk solids between 12% and 14% (including a minimum of milk fat between 2.5% and 3.5%). “Milk solids” refers to the addition of milk ingredients in their natural proportions, except that milk fat may be added, or removed. Where required by the competent authority, a minimum content of cocoa butter plus milk fat may also be set.

The following process may be used to produce 50 g of milk chocolate with CCM using the ingredients of sweetened cocoa mass bar (30 g), skimmed CCM powder (10 g), cocoa butter (5 g), powdered sugar (5 g). This recipe may be scaled to produce larger quantities. An alternative composition that may be used, with adjustments to meet the desired end properties, is 40% cocoa mass, 10% skimmed CCM powder, 20% cocoa butter and 30% powdered sugar.

In a double boiler, melt the sweetened cocoa mass bar and cocoa butter together until fully combined and smooth (about 5-10 minutes). The temperature should be kept below 45 °C (113 °F) to avoid burning. Gradually add the CCM powder to the melted mixture, stirring continuously until fully dissolved and smooth. To ensure a smooth texture and a glossy finish, the chocolate is tempered by cooling the mixture to about 27-28 °C (80-82 °F), then reheating to 31-32 °C (88-90 °F). This takes about 10-15 minutes. The melted chocolate is then spread onto a cool surface (such as marble or chilled stainless steel) and moved around with a spatula. Alternatively, a bowl of chocolate may be placed in a cool water bath while stirring constantly. With either approach, the key is to avoid any moisture getting into the chocolate. The tempered chocolate is then poured into molds and tapped gently to remove air bubbles. The chocolate is allowed to set at room temperature or placed in a refrigerator until solid. Once solidified, the chocolate can be removed from the molds and stored in a cool, dry place.

Quality and Taste: The use of cell-cultivated milk in chocolate results in a smoother texture, richer flavor profile, and enhanced creaminess compared to chocolates made with conventional milk. Moreover, the absence of lactose in cell-cultivated milk enhances the chocolate's overall taste and makes it accessible to lactose-intolerant consumers. Similar benefits can be obtained using CCM in the production of other confections such as caramels, fudge, toffee, pralines, and sweetmeats.

### Example 14: Ice Cream and Milk-Based Desserts

Formulation Incorporation: CCM is integrated into ice cream formulations as a primary dairy component. The milk is mixed with cream, sugars, stabilizers, and flavors in specific ratios to create a smooth and creamy ice cream base.

The following is a sample recipe for producing panna cotta, a classic Italian custard dessert, using ingredient including CCM, granulated sugar, gelatin (powdered) and water.

In a vessel, combined CCM and sugar and heat over medium heat, stirring constantly to prevent scorching. Continue heating until the sugar is fully dissolved, taking care not to allow the mixture to boil. Remove the milk mixture from heat. Soften the gelatin in cold water and add to the hot milk mixture, stirring until the gelatin is completely dissolved, with no lumps remaining. Flavored extract, e.g., vanilla or other flavor, may be added for flavor. Pour the mixture into molds at room temperature to cool slightly before placing them in a refrigerator to chill until set-typically about 2-4 hours.

Texture and Nutritional Benefits: Ice cream and desserts made with CCM exhibits excellent creaminess, similar to premium dairy ice cream. Additionally, it boasts a reduced saturated fat content and offers a higher protein content, contributing to its nutritional appeal. As with the chocolate production process described above, ice cream can be made using cell-cultivated milk with no- or reduced-lactose, allowing these confections to be enjoyed by lactose-intolerant consumers.

### Example 15: Dairy Product Manufacturing-Cheese

Cheese Production: Cell-cultivated milk serves as the foundational milk source for cheese production. It undergoes the same processes as traditional milk, including curdling, cutting, and maturation, resulting in a variety of cheeses with unique flavors and textures. The cell-cultured milk may be used in place of traditional milk or cream for a wide range of conventional and specialty cheese processing, including hard and soft cheeses, curds, whey, ethnic dairy products such as paneer (Indian cottage cheese), Queso fresco, Cotija (Mexican cheeses), Labneh (Middle Eastern strained yogurt), Ayran (Turkish yogurt drink), Kumis (fermented mare's milk in Central Asia), artisanal cheeses such as Gruyère and Roquefort, smoked or spiced cheeses, and more.

Soft cheese: Fresh or unripened cheese has a high moisture content of about 75%. It is made by removing the whey from soured, skimmed cell cultivated milk (CCM). The CCM is usually coagulated by souring using the following sample process:

CCM, which may be liquid or a mixture of liquid and powdered CCM, is heated in a vessel to about 80 °C to 90 °C to boiling then the heat is removed. Vinegar acid (25%) is added gradually (drop by drop) to the warm milk, which curdles quickly at higher temperatures. The curd is collected by straining the curdled milk through a strainer, which may be cheesecloth or other fine-mesh strainer. The strained curd may be poured into a conventional cheese mold. The curd may be optionally pressed by placing a plate or other flat surface on top of the curd and applying pressure. The resulting soft cheese may be consumed fresh or may be stored in a refrigerator.

Cream Cheese: Using the ingredients of CCM, a small amount of mild acid such as white vinegar or lemon juice, and salt (optional), cream cheese can be produced by heating the CCM in a vessel over medium heat until it starts to steam and small bubbles form around the edges (about 80 °C/176 °F). The milk should not be allowed to boil. To curdle the CCM, remove the vessel from heat and slowly add the vinegar or lemon juice while stirring gently. The milk will curdle and separate into curds (solid) and whey (liquid). If it doesn't curdle completely, more vinegar or lemon juice can be added. The curds are then strained using fine-mesh strainer or colander with a clean cheesecloth or a thin kitchen towel. Pour the curdled milk into the strainer to separate the whey from the curds and allow to drain for about 10-15 minutes. The drained curds are then transferred into a blender or food processor. Salt may be added if desired and the curds are blended until smooth and creamy. If the blended curds are too thick, a small amount of whey can be blended into the mixture. These steps may be repeated as needed to achieve the desired consistency. The resulting cream cheese is then transferred into a storage container and may be used immediately or stored in a refrigerator. Preferably, the product should be used within 5-7 days.

Hard and semi-hard Cheese: CCM (either liquid or reconstituted powder) can be used to produce various hard and semi-hard cheeses. Additional ingredients include rennet, which is widely commercially available, salt, and starter culture. As is known in the art, starter cultures, which are commercially available, vary depending on the type of cheese to be produced. For example, starter compositions containing Lactococcus lactis ssp. lactis and Lactococcus lactis ssp. cremoris are commonly used for cheeses including Cheddar, Colby, Gouda, Edam, and Monterey. Starter cultures for Swiss, Emmental, Gruyere, Samso, Fontina cheese include Lactococcus lactis ssp. lactis biovar. diacetylactis, Leuconostoc lactis ssp. cremoris, Lactobacillus delbrueckii ssp. bulgaricus, Lactobacillus delbrueckii ssp. lactis, Lactobacillus casei ssp. casei, Lactobacillus helveticus, Streptococcus thermophilus, Propionibacterium freudenreichii, and Propionibacterium shermanii. Biovariant specie diactelylactis, also called L. lactis citrate+, produces CO₂ and a buttery flavor compound (diacetyl) from normal milk constituent citrate. A weak acid producer Leuconostoc mesentroides ssp. cremoris also produces diacetyl and CO₂. The flavor compound (diacetyl) is essential in fresh cheese production. They are used in cheese varieties, such as soft-ripened, Cheddar, most washed, and fresh cheeses. Other culture starters are known in the art for use in production of many types of hard and semi-hard cheeses.

For preparation of hard cheese, the CCM is heated in a vessel to 32 °C (90 °F) for mesophilic cheese or 38 °C (100 °F) for thermophilic cheese. A food-grade thermometer should be used for accuracy. The starter culture is sprinkled over the surface of the milk and allowed to rehydrate for 1-2 minutes. The mixture is gently stirred for about 30 seconds then allowed to sit for 30-60 minutes to acidify. The rennet is diluted in cool, chlorine-free water (ratio of about 1:25 rennet to water) and stirred gently into the milk in an up-and-down motion. The mixture is allowed to sit undisturbed for 30-60 minutes until it sets into a firm gel (clean break stage). A long blade is used to cut the curd into small cubes (about 1 cm) which are allowed to rest for about 5 minutes. The cubed curds are slowly heated in the vessel while stirring gently, increasing the temperature to 38-40 °C (100-104 °F) over 30-40 minutes. This step helps remove whey and firm up the curds. After heating, the whey is drained by lining a colander with cheesecloth and pouring the curds into it. The whey may be saved for other uses if desired. Salt is sprinkled evenly over the curds and mixed gently. Salt helps flavor the cheese and acts as a preservative. The curds are pressed into a cheese mold lined with cheesecloth by applying a weight of 5-10 kg (10-20 lbs) to the top of the curds for about 12-24 hours. The mold is flipped occasionally to ensure even pressing. The cheese is next removed from the mold and allowed to air dry at room temperature for 1-2 days until the surface is dry to the touch. The cheese may optionally be waxed to prevent drying out, and aged in a cool, humid environment (10-12 °C/50-55 °F, 80-85% humidity) for 1-3 months or longer, depending on the type of cheese and desired flavor.

### Example 16: Dairy Product Manufacturing-Yogurt and Butter

In yogurt and cream production, cell-cultivated milk serves as the primary dairy ingredient. The resulting yogurt exhibits a smooth and creamy texture, while creams made from cell-cultivated milk offer similar functionalities and flavors to those made with conventional dairy.

Yogurt: The following provides a sample process for yogurt production using cell-cultivated milk. Ingredients include liquid cell-cultivated milk (CCM-L), skimmed cell-cultivated milk powder (CCM-P) and lactose-free cell-cultivated milk (LFCCM) in a ratio of about 15:2:0.5-1.0 and a small quantity of starter culture freeze-dried powder comprising Lactobacillus acidophilus, bifidobacterium, Streptococcus thermophilus. As is known in the art, a variety of different bacteria starter culture combinations may be used, including, but not limited to, Lactobacillus delbrueckii subsp. Bulgaricus and S. thermophilus. The milks are combined in a vessel and heated to 85 °C or higher and maintained at that temperature for 3-5 minutes, then cooled to about 35 °C to 45 °C. The starter culture is mixed into the milk in the vessel and left to ferment. The time required for the cell cultivated milk to turn sour depends on the temperature. At 35 °C to 45 °C the incubation period takes about 15 to 20 hours. Ideally, incubation will be performed within an insulated chamber or other heated environment to maintain a consistent temperature. The yogurt will be ready for consumption once the incubation period is finished. Flavorings, sweeteners (sugar, honey, syrups, etc.), fruits (pureed, jellied, crushed, etc.) may be added for taste.

In a variation on the above process, yogurt may be made starting with CCM powder by adding water to the CCM powder according to the instructions on the package for making milk, adding 10 to 15% extra milk powder. Dissolve the CCM-P in water, heat to boiling point and allow to cool to 45 °C. Stir in 1-3 tablespoons of fresh yogurt or a yogurt culture per liter of milk. Cover the vessel and place it in a warm, insulated place. After 15-20 hours (at 35 °C to 45 °C), the yogurt will be firm and concentrated, ready for consumption.

Butter: CCM can be used to produce butter using the following process. CCM is processed as described above to create a heavy cream with 35-40% fat. If the cream has been refrigerated, it should be allowed to sit at room temperature for about 30 minutes. This step improves the efficiency of churning. The room temperature heavy cream is placed in a mixing vessel (bowl or vat) and whipped at medium high speed using an electric mixer or food processor until it separates into butterfat (solid) and buttermilk (liquid). This takes about 8-15 minutes. Initially, a whipped cream will be formed, after which separation will occur. The mixture is strained through a fine mesh strainer or cheesecloth to separate the buttermilk from the butterfat. The buttermilk may be used for baking or cooking. The butterfat should be rinsed in cold water, then rinsed and kneaded gently to remove any remaining buttermilk. Rinsing may be repeated until the water runs clear. A fine salt may be optionally kneaded into the butterfat to taste. The resulting CCM butter can be packed into a container or mold, covered and stored in a refrigerator or freezer.

### Example 17: Dairy Product Manufacturing-Liquid Milk Products

Condensed and evaporated milk products are widely used in baking and cooking. In general, concentrated milks are obtained by removing part of water through evaporation from whole or full-cream milk, partly skimmed milk, or skimmed milk with or without addition of sugar. Both evaporated and sweetened condensed milk are made by heating fresh cell cultivated milk until about 60 percent of the water content has been removed.

To make sweetened condensed milk, a mixture of CCM and skimmed CCM powder is combined in a vessel for heating. The vessel should preferably have a wide bottom and be filled so that the liquid is relatively shallow so that a large surface area of the milk is exposed to expedite evaporation. The heat should be set to medium-high, i.e., about 60 °C to 70 °C. Granulated sugar is added gradually to the CCM, stirring to ensure complete dissolution, to produce a mixture with about 40% sugar. The mixture is brought to a rolling boil, stirring thoroughly to prevent sticking or burning. Once the milk reaches a boil, the heat is reduced to medium while stirring continuously for 2-3 minutes to prevent the milk from boiling over. After 2-3 minutes, the heat is increased to medium-high, maintaining a steady boil. Constant stirring is essential to prevent the milk from sticking to the bottom of the pan or boiling over. The mixture should boil continuously, ensuring it remains bubbling throughout the process. This step will take approximately 15-25 minutes. After 15-17 minutes, the milk should begin to thicken. Cooking can be continued until the desired consistency is achieved, usually around 1 hour. After cooking, the milk is allowed to cool to room temperature. It will continue to thicken further as it cools.

Evaporated milk can be produced in a similar manner to the condensed milk by removing about 60% of the water content from the CCM. a mixture of CCM and skimmed CCM powder are combined in a vessel for heating. The mixture is heated until the liquid's temperature is between 55 °C and 60 °C. This lower temperature (relative to the condensed milk process) helps prevent the milk from developing a “cooked” flavor. The liquid is continuously heated and stirred until it reduces in volume. This process can take 30 minutes to 1 hour, depending on the quantity of milk and the heat level.

### Example 18: Specific Cultivation Techniques

Enhanced Cell Selection: An important aspect of the inventive methods lies in the meticulous selection and isolation of mammary epithelial cells with exceptional milk-producing capabilities. This selection process ensures a superior starting cell population, contributing to higher milk yield and quality during cultivation.

Proprietary Culture Medium: The inventive step involves the formulation of a nutrient-rich culture medium optimized to support robust cell growth and efficient milk synthesis. This unique medium composition, comprising a tailored blend of growth factors and nutrients, contributes significantly to maximizing milk production.

### Quality Enhancement Methods:

Precise Environmental Control: The inventive step includes a sophisticated bioreactor system equipped with precise control mechanisms regulating temperature, pH, and oxygen levels. This tight control over environmental factors ensures optimal conditions for cell growth and milk production, resulting in consistent and high-quality cell-cultivated milk.

Monitoring and Harvesting Optimization: Another novel aspect involves continuous monitoring techniques to assess cell growth parameters and determine the optimal harvesting stage. This precise timing ensures maximum milk yield while maintaining the integrity and viability of the cultivated cells.

### Comparative Analysis:

Environmental Sustainability: The innovative aspect extends beyond the technical processes to encompass a comprehensive comparative analysis highlighting the environmental sustainability of cell-cultivated milk. This includes data showcasing reduced carbon footprint, land usage, and water consumption compared to traditional dairy farming methods.

Quality and Nutritional Superiority: Comparative studies demonstrate the superiority of products made with cell-cultivated milk in terms of taste, texture, and nutritional benefits compared to those using traditional milk sources add to the inventive step.

Each application described herein benefits from the unique properties of cell-cultivated milk, providing superior taste, texture, and nutritional advantages over products made with traditional milk sources. The incorporation of cell-cultivated milk in these food items showcases its versatility and potential to revolutionize the quality and sustainability of various dairy and confectionery products, as described in the patent application.

The utilization of cell-cultivated milk in the production of confectionery and dairy products represents a paradigm shift in the food industry. This innovative method harnesses advanced cell culture techniques to provide a sustainable, ethical, and scalable solution to the challenges posed by traditional milk sourcing.

By cultivating milk cells in controlled environments, the inventive approach not only ensures a consistent and high-quality milk supply but also addresses critical concerns regarding animal welfare, environmental impact, and resource sustainability. The versatility of cell-cultivated milk in applications across a spectrum of products, including chocolate and other confections, e.g., fudge, caramel and toffee, ice cream, cheese, yogurt, and cream, highlights its adaptability and potential to revolutionize food production.

Furthermore, the use of cell-cultivated milk introduces a new standard of excellence in taste, texture, and nutritional value, offering consumers a superior alternative to conventionally sourced milk. The reduced carbon footprint, absence of animal exploitation, and enhanced production efficiency signify a step towards a more environmentally conscious and ethically responsible future for the food industry. The incorporation of cell-cultivated milk in confectionery and dairy products not only presents a groundbreaking technological advancement but also embodies a transformative approach to sustainable and ethical food production, benefiting both producers and consumers alike.

### Example 19: Non-Food Products-Pharmaceuticals and Cosmetics

Although milk is well known for its use as a raw material in the food industry, it is also widely used in the pharmaceutical and cosmetic industries due to its considerable biological potential. These natural products are especially rich in proteins, such as casein, β-lactoglobulin, α-lactalbumin, lactoferrin, immunoglobulins, lactoperoxidase, lysozyme, and growth factors, and possess various antibacterial, antifungal, antiviral, anticancer, antioxidant, immunomodulatory properties, etc. The milk-based products can be used in dietary supplementation and for performing immunomodulatory functions; they can enhance the effects of certain drugs and can have a suppressive effect on pathogenic microorganisms. Milk products are widely used in the treatment of dermatological diseases for promoting the healing of chronic wounds, hastening tissue regeneration, and the treatment of acne vulgaris or plaque psoriasis. They are also increasingly regarded as active ingredients that can improve the condition of the skin by reducing the number of acne lesions and blackheads, regulating sebum secretion, ameliorating inflammatory changes as well as bestowing a range of moisturizing, protective, toning, smoothing, anti-irritation, whitening, soothing, and antiaging effects.

Cell selection as described above can be used to mimic the compositions and bioactive protein components of the milk of different species for specific uses. Recent years have seen a growth in interest in products derived from milk- and colostrum from goats, sheep, cows, buffalo, donkeys, and horses for use in the cosmetics and pharmaceutical industries. The milk protein most commonly used as a supplement is lactoferrin, possibly due to its broad spectrum of proven biological properties. Lactoferrin has been found to induce a significant improvement in the skin condition of patients with psoriasis and acne vulgaris, including a reduction in the number of inflammatory lesions. Lactoferrin is also used in other health supplements and infant formulas. Additionally, probiotics, which are discussed above, can be derived from fermented cell-cultured milk.

Milk-based cosmetics for topical administration, e.g., lotions, creams, and make-up, are widely available, touted as a natural source of lactic acid, hyaluronic acid, fatty acids, vitamin A, and more. Milk is generally considered to be good for all skin types since it is a non-comedogenic, gentle exfoliant. The use of cell-cultured milk enables engineering of the contents of a key active ingredient of milk beauty products to reduce or moderate potential irritants. Importantly, the ethical, non-animal sourcing of the cell-cultured milk ingredient addresses a common objection to many cosmetics.

### Example 20: Non-Food Products-Plastics and Adhesives

Milk plastic, also known as casein plastic, has been known for well over 100 years. While no longer widely in use after the adoption of more durable synthetic (petroleum-based) plastics, milk plastic has the advantage of being more biodegradable than most modern plastics. Depending on the formulation, milk plastic can decompose in as little as 30 days. Milk plastic based on CCM could provide a solution to growing environmental concerns about plastics pollution by providing a modified, more easily biodegradable plastic material. An exemplary process for fabricating milk plastic with epoxy resin uses the materials listed in Table 5 below:

TABLE 5

| | |
|---|---|
| Epoxy Resin (A&B) | 1 mL resin |
| Silicone Mold | 1 |
| Mixing eppi tubes | 4 |
| Pipettes | 2 |
| Heat Gun (To remove air bubbles) | 1 |
| MILK | |
| Liquid milk | 300 ul milk + 1 mL resin |
| Powdered milk | 800 ul A + B + 200 ul milk |

In a well-ventilated area and wearing appropriate protective gear, measure equal parts of epoxy resin and hardener according to the manufacturer's instructions. Add a mixture of liquid and powdered CCM to the mixed resin, stirring well until the milk is evenly distributed. Slowly pour the resin into a silicone mold. Pouring in layers can help create depth and a more natural stone-like appearance. Use a heat gun to remove any air bubbles that rise to the surface, being careful not to overheat, as this can cause the resin to cure too quickly or burn. Allow the resin to dry as per the manufacturer's instructions, typically 24-48 hours. Ensure the mold is kept level during this time to maintain the desired shape. After drying, remove the plastic pieces from the mold.

Scaling of the recipe and process for larger batches, and to create a product with desired characteristics, will be within the level of skill in the art based on the foregoing disclosure.

Casein glue can be produced using CCM using the materials listed in Table 6 below:

TABLE 6

| Ingredients | Quantity | |
|---|---|---|
| Skimmed CC milk powder | 2.5 | ml |
| CC milk | 2.5 | ml |
| Sodium bicarbonate | 0.05 | g |
| White Vinegar | | |
| Casein from Skimmed milk | 0.41 | g |
| Casein from invitro milk | 0.7 | g |
| Hot plate | 1 | |
| Thermometer | 1 | |
| Glass Stirrer | 1 | |
| Filter Paper | 1 | |
| Beaker | 2 | |
| Water | 15 | ul |

Pieces of paper

Pour 2.5 mL of cell cultivated milk into a beaker and add 50 mL of white vinegar (5% acetic acid) to the milk. Place the beaker on a hot plate and gently heat the mixture while stirring with a thermometer. The mixture should be observed carefully, and the temperature record when turbidity (chunks or curds) appears in the liquid. Folded paper towels, a fine-mesh screen, or paper filters can be used to filter the curdled mixture by placing it in a funnel. The liquid whey should pass through the filter, leaving the curds. The curds are then scraped from the filter back into the beaker. Add about 0.05 g of sodium bicarbonate (NaHCO₃) to the curds in the beaker and stir the mixture thoroughly. The consistency of the mixture can be adjusted by gradually adding drops of water to the mixture, stirring intermittently, until the consistency of white glue is achieved. The resulting glue can be used to fasten items such as paper, wood, textiles, and more.

The foregoing describes embodiments of processes for and applications of in vitro milk production. Although specific example embodiments have been described, it will be evident that various modifications and changes may be made to these embodiments without departing from the broader scope of the invention. Accordingly, the detailed description is not to be taken in a limiting sense, and the scope of various embodiments is defined only by the appended claims, along with the full range of equivalents to which such claims are entitled.

1. A method for producing confectionery and dairy products, comprising:
cultivating milk cells in an in vitro nutrient-rich medium under controlled conditions to produce a cell-cultivated milk product;
harvesting the cell-cultivated milk product at an optimal stage of cultivation according to an intended food product; and
incorporating the cell-cultivated milk product the intended food product, wherein the food product is one of more of chocolate, confections, ice cream, cheese, yogurt, and cream.

2. The method of claim 1, wherein the cultivated milk cells are selected based on their optimal milk-producing capabilities for purposes of the intended food product.

3. The method of claim 1, wherein the nutrient-rich medium comprises a blend of growth factors, nutrients, and conditions conducive to enhancing milk quality and yield.

4. The method of claim 1, wherein incorporation of the cell-cultivated milk product in the intended food product results in a food product with enhanced nutritional profiles compared to a food product made with traditional milk sources.

5. A system for the large-scale production of confectionery and dairy products utilizing cell-cultivated milk, comprising:
a bioreactor configured for cultivating milk cells in vitro in a nutrient-rich medium to produce a cell-cultivated milk product;
a processing unit configured for integrating the cell-cultivated milk product into an intended food product; and
a quality control unit configured for testing and ensuring consistency and quality of one or more of the cell-cultivated milk product and the food product.

6. The system of claim 5, further comprising monitoring devices and software algorithms to regulate and optimize cultivation conditions within the bioreactor.

7. The system of claim 5, wherein the processing unit further comprises tools and machinery configured to producing a specific food product, wherein the specific food product is one of more of chocolate, confections, ice cream, cheese, yogurt, and cream.

8. A method for producing a milk ingredient for milk-based products, comprising:
cultivating milk cells in an in vitro nutrient-rich medium under controlled conditions to produce a cell-cultivated milk ingredient
harvesting the cell-cultivated milk ingredient at an optimal stage of cultivation according to an intended milk-based product; and
combining the cell-cultivated milk ingredient with additional components of the milk-based product to form a final milk-based product.

9. The method of claim 8, wherein the milk-based product is a food product and the cultivated milk cells are selected based on their optimal milk-producing capabilities for purposes of the intended food product.

10. The method of claim 9, wherein the nutrient-rich medium comprises a blend of growth factors, nutrients, and conditions conducive to enhancing milk quality and yield.

11. The method of claim 9, wherein incorporation of the cell-cultivated milk product in the intended food product results in a food product with enhanced nutritional profiles compared to a food product made with traditional milk sources.

12. The method of claim 9, wherein the food product is one of more of chocolate, candy, ice cream, cheese, yogurt, and cream.

13. The method of claim 8, wherein the milk-based product is a pharmaceutical or cosmetic product.

14. The method of claim 8, wherein the milk-based product is a plastic or an adhesive product.
